SEQUENCE LISTING (61 sequences)

SEQ ID NO: 1 - protein, 16 aa, Artificial Sequence
  Description of Artificial Sequence Synthetic peptide
WDCLDNXIGRRQCVXL

SEQ ID NO: 2 - protein, 16 aa, Artificial Sequence
  Description of Artificial Sequence Synthetic peptide
WDCLDNRIGRRQCVKL

SEQ ID NO: 3 - protein, 19 aa, Artificial Sequence
  Description of Artificial Sequence Synthetic peptide
GGSWDCLDNRIGRRQCVKL

SEQ ID NO: 4 - protein, 20 aa, Artificial Sequence
  Description of Artificial Sequence Synthetic peptide
GGGSWDCLDNRIGRRQCVKL

SEQ ID NO: 5 - protein, 16 aa, Artificial Sequence
  Description of Artificial Sequence Synthetic peptide
WDCLDNXIGRRQCVKL

SEQ ID NO: 6 - protein, 14 aa, Artificial Sequence
  Description of Artificial Sequence Synthetic peptide
WDCLDRPGRRQCVK

SEQ ID NO: 7 - protein, 16 aa, Artificial Sequence
  Description of Artificial Sequence Synthetic peptide
WDCLDNKIGRRQCVRL

SEQ ID NO: 8 - protein, 12 aa, Artificial Sequence
  Description of Artificial Sequence Synthetic peptide
CLDNRIGRRQCV

SEQ ID NO: 9 - protein, 15 aa, Artificial Sequence
  Description of Artificial Sequence Synthetic peptide
DCLDNRIGRRQCVKL

SEQ ID NO: 10 - protein, 16 aa, Artificial Sequence
  Description of Artificial Sequence Synthetic peptide
WDCLDNRIGKRQCVRL

SEQ ID NO: 11 - protein, 16 aa, Artificial Sequence
  Description of Artificial Sequence Synthetic peptide
WDCLDNRIGXRQCVXL

SEQ ID NO: 12 - protein, 15 aa, Artificial Sequence
  Description of Artificial Sequence Synthetic peptide
WDCLDNRIGRRQCVK

SEQ ID NO: 13 - protein, 18 aa, Artificial Sequence
  Description of Artificial Sequence Synthetic peptide
WDCLVDRPGRRQCVRLEK

SEQ ID NO: 14 - protein, 19 aa, Artificial Sequence
  Description of Artificial Sequence Synthetic peptide
WDCLVDRPGRRQCVRLERK

SEQ ID NO: 15 - protein, 18 aa, Artificial Sequence
  Description of Artificial Sequence Synthetic peptide
WDCLVDRPGRRQCVKLER

SEQ ID NO: 16 - protein, 15 aa, Artificial Sequence
  Description of Artificial Sequence Synthetic peptide
WDCLDNRIGKRQCVR

SEQ ID NO: 17 - protein, 16 aa, Artificial Sequence
  Description of Artificial Sequence Synthetic peptide
WDCLDNRIGKRQCVRA

SEQ ID NO: 18 - protein, 16 aa, Artificial Sequence
  Description of Artificial Sequence Synthetic peptide
WDCLDNXIGRRQCVXL

SEQ ID NO: 19 - protein, 16 aa, Artificial Sequence
  Description of Artificial Sequence Synthetic peptide
WDCLDNRIGRRQCVKL

SEQ ID NO: 20 - protein, 19 aa, Artificial Sequence
  Description of Artificial Sequence Synthetic peptide
GGSWDCLDNRIGRRQCVKL

SEQ ID NO: 21 - protein, 16 aa, Artificial Sequence
  Description of Artificial Sequence Synthetic peptide
WDCLDNXIGRRQCVKL

SEQ ID NO: 22 - protein, 14 aa, Artificial Sequence
  Description of Artificial Sequence Synthetic peptide
WDCLDRPGRRQCVK

SEQ ID NO: 23 - protein, 16 aa, Artificial Sequence
  Description of Artificial Sequence Synthetic peptide
WDCLDNKIGRRQCVRL

SEQ ID NO: 24 - protein, 12 aa, Artificial Sequence
  Description of Artificial Sequence Synthetic peptide
CLDNRIGRRQCV

SEQ ID NO: 25 - protein, 15 aa, Artificial Sequence
  Description of Artificial Sequence Synthetic peptide
DCLDNRIGRRQCVKL

SEQ ID NO: 26 - protein, 16 aa, Artificial Sequence
  Description of Artificial Sequence Synthetic peptide
WDCLDNRIGKRQCVRL

SEQ ID NO: 27 - protein, 16 aa, Artificial Sequence
  Description of Artificial Sequence Synthetic peptide
WDCLDNRIGXRQCVXL

SEQ ID NO: 28 - protein, 15 aa, Artificial Sequence
  Description of Artificial Sequence Synthetic peptide
WDCLDNRIGRRQCVK

SEQ ID NO: 29 - protein, 18 aa, Artificial Sequence
  Description of Artificial Sequence Synthetic peptide
WDCLVDRPGRRQCVRLEK

SEQ ID NO: 30 - protein, 19 aa, Artificial Sequence
  Description of Artificial Sequence Synthetic peptide
WDCLVDRPGRRQCVRLERK

SEQ ID NO: 31 - protein, 18 aa, Artificial Sequence
  Description of Artificial Sequence Synthetic peptide
WDCLVDRPGRRQCVKLER

SEQ ID NO: 32 - protein, 20 aa, Artificial Sequence
  Description of Artificial Sequence Synthetic peptide
GGGSWDCLDNRIGRRQCVKL

SEQ ID NO: 33 - protein, 15 aa, Artificial Sequence
  Description of Artificial Sequence Synthetic peptide
WDCLDNRIGKRQCVR

SEQ ID NO: 34 - protein, 16 aa, Artificial Sequence
  Description of Artificial Sequence Synthetic peptide
WDCLDNRIGKRQCVRA

SEQ ID NO: 35 - protein, 16 aa, Artificial Sequence
  Description of Artificial Sequence Synthetic peptide
CWDLDNRIGRRQVCKL

SEQ ID NO: 36 - protein, 16 aa, Artificial Sequence
  Description of Artificial Sequence Synthetic peptide
CWDLDNRIGRRQVCKL

SEQ ID NO: 37 - protein, 20 aa, Artificial Sequence
  Description of Artificial Sequence Synthetic peptide
GGGSWDCLDNRIGRRQCVKL

SEQ ID NO: 38 - protein, 18 aa, Artificial Sequence
  Description of Artificial Sequence Synthetic peptide
GSWDCLDNRIGRRQCVKL

SEQ ID NO: 39 - protein, 17 aa, Artificial Sequence
  Description of Artificial Sequence Synthetic peptide
SWDCLDNRIGRRQCVKL

SEQ ID NO: 40 - protein, 14 aa, Artificial Sequence
  Description of Artificial Sequence Synthetic peptide
CLDNRIGRRQCVKL

SEQ ID NO: 41 - protein, 19 aa, Artificial Sequence
  Description of Artificial Sequence Synthetic peptide
GGGSWDCLDNRIGRRQCVK

SEQ ID NO: 42 - protein, 18 aa, Artificial Sequence
  Description of Artificial Sequence Synthetic peptide
GGSWDCLDNRIGRRQCVK

SEQ ID NO: 43 - protein, 17 aa, Artificial Sequence
  Description of Artificial Sequence Synthetic peptide
GSWDCLDNRIGRRQCVK

SEQ ID NO: 44 - protein, 16 aa, Artificial Sequence
  Description of Artificial Sequence Synthetic peptide
SWDCLDNRIGRRQCVK

SEQ ID NO: 45 - protein, 15 aa, Artificial Sequence
  Description of Artificial Sequence Synthetic peptide
WDCLDNRIGRRQCVK

SEQ ID NO: 46 - protein, 14 aa, Artificial Sequence
  Description of Artificial Sequence Synthetic peptide
DCLDNRIGRRQCVK

SEQ ID NO: 47 - protein, 13 aa, Artificial Sequence
  Description of Artificial Sequence Synthetic peptide
CLDNRIGRRQCVK

SEQ ID NO: 48 - protein, 18 aa, Artificial Sequence
  Description of Artificial Sequence Synthetic peptide
GGGSWDCLDNRIGRRQCV

SEQ ID NO: 49 - protein, 17 aa, Artificial Sequence
  Description of Artificial Sequence Synthetic peptide
GGSWDCLDNRIGRRQCV

SEQ ID NO: 50 - protein, 16 aa, Artificial Sequence
  Description of Artificial Sequence Synthetic peptide
GSWDCLDNRIGRRQCV

SEQ ID NO: 51 - protein, 15 aa, Artificial Sequence
  Description of Artificial Sequence Synthetic peptide
SWDCLDNRIGRRQCV

SEQ ID NO: 52 - protein, 14 aa, Artificial Sequence
  Description of Artificial Sequence Synthetic peptide
WDCLDNRIGRRQCV

SEQ ID NO: 53 - protein, 13 aa, Artificial Sequence
  Description of Artificial Sequence Synthetic peptide
DCLDNRIGRRQCV

SEQ ID NO: 54 - protein, 12 aa, Artificial Sequence
  Description of Artificial Sequence Synthetic peptide
CLDNRIGRRQCV

SEQ ID NO: 55 - protein, 17 aa, Artificial Sequence
  Description of Artificial Sequence Synthetic peptide
GGGSWDCLDNRIGRRQC

SEQ ID NO: 56 - protein, 16 aa, Artificial Sequence
  Description of Artificial Sequence Synthetic peptide
GGSWDCLDNRIGRRQC

SEQ ID NO: 57 - protein, 15 aa, Artificial Sequence
  Description of Artificial Sequence Synthetic peptide
GSWDCLDNRIGRRQC

SEQ ID NO: 58 - protein, 14 aa, Artificial Sequence
  Description of Artificial Sequence Synthetic peptide
SWDCLDNRIGRRQC

SEQ ID NO: 59 - protein, 13 aa, Artificial Sequence
  Description of Artificial Sequence Synthetic peptide
WDCLDNRIGRRQC

SEQ ID NO: 60 - protein, 12 aa, Artificial Sequence
  Description of Artificial Sequence Synthetic peptide
DCLDNRIGRRQC

SEQ ID NO: 61 - protein, 11 aa, Artificial Sequence
  Description of Artificial Sequence Synthetic peptide
CLDNRIGRRQC

(19) United States
(12) Patent Application Publication
(10) Pub. No.: US 2009/0131317 A1
(43) Pub. Date: May 21, 2009

(54) COMPOUNDS AND PEPTIDES THAT BIND THE TRAIL RECEPTOR

(75) Inventors: Yvonne M. Angell, San Carlos, CA (US); Ashok Bhandari, Cupertino, CA (US); Jennifer Green, Belmont, CA (US); Peter J. Schatz, Cupertino, CA (US); Christopher P. Holmes, Saratoga, CA (US)

Correspondence Address: DARBY & DARBY P.C., P.O. BOX 770, Church Street Station, New York, NY, 10008-0770, US

(73) Assignee: AFFYMAX, INC., Palo Alto, CA (US)

(21) Appl. No.: 12/144,584

(22) Filed: Jun. 23, 2008

Related U.S. Application Data
(60) Provisional application No. 60/945,780, filed on Jun. 22, 2007.

(51) Int. Cl. A61K 38/16; C07K 14/00; C07K 7/08; A61P 35/00; A61P 11/06; A61K 38/10

(52) U.S. Cl. 514/12; 530/326; 530/327; 530/324; 514/13; 514/14

(57) ABSTRACT

The present invention relates to peptides and compounds that bind to a TRAIL receptor or otherwise act as a TRAIL receptor agonist, as well as methods of treating human diseases using the same. In addition, methods of synthesizing the peptides and compounds described herein are provided by the present invention.

### CROSS-REFERENCE TO PRIOR APPLICATION

The present application claims the benefit under 35 U.S.C. § 119(e), of U.S. Provisional Application No. 60/945,780, filed on Jun. 22, 2007, which is hereby incorporated herein by reference in its entirety.

### FIELD OF THE INVENTION

The present invention relates to peptides and compounds that bind to a TRAIL receptor or otherwise act as a TRAIL receptor agonist or antagonist, as well as methods of treating human diseases using the same. In addition, methods of preparing and synthesizing the peptides and compounds described herein are provided by the present invention.

### BACKGROUND OF THE INVENTION

TRAIL is a type II transmembrane protein that is a member of the tumor necrosis factor (TNF) gene superfamily and contains an extracellular region that can be proteolytically cleaved to release the soluble molecule [Wiley et al., (1995) Immunity 3: 673-682; Pitti et al., (1996), J. Biol. Chem. 271(22):12687-12690; Ashkenazi and Dixit (1998) Science 281: 1305-1308; the disclosure(s) of the DNA sequence(s) for TRAIL ligand in Wiley et al. (1995) and Pitti et al., (1996) are expressly incorporated herein by reference in their entirety]. In its active form, TRAIL is a zinc-coordinated trimer (Hymowitz et al. Mol. Cell, 4, 563-571 (1999)) that is involved in modulation of apoptosis as well as inflammatory cascades.

Five receptors for TRAIL have been identified including TRAIL R1 (DR4) [Pan et al., (1997) Science 276: 111-113], TRAIL R2 (DR5/KILLER) [Pan et al., (1997) Science 277: 815-818; Sheridan et al., (1997) Science 277: 818-821], TRAIL R3 (TRID/DcR1) [Pan et al., (1997) Science 277: 815-818; Degli-Esposti et al., (1997) Immunity 7: 821-830; Sheridan et al., (1997) Science 277: 818-821], TRAIL R4 (DcR2) [Degli-Esposti et al., (1997) Immunity 7: 821-830; Marsters et al., (1997) Current Biology 7: 1003-1006], and osteoprotegerin [Emery et al., (1997) Journal of Biological Chemistry 273: 14363-14367]. TRAIL R1 and R2 are each single transmembrane receptors arranged as a homotrimeric complex on the cell membrane. The extracellular domain of the receptors is characterized by concatenated cysteine-rich domains (CRDs) [Bazan, J. F. Curr. Biol. 3, 603-606 (1993)] that are responsible for ligand binding. Both TRAIL R1 and TRAIL R2 contain a conserved, cytoplasmic death domain.

Upon binding to TRAIL R1 and R2, TRAIL triggers cell apoptosis independently of the p53 tumor-suppressor gene through the “extrinsic” pathway of apoptosis [Reviewed in Ashkenazi et al. J. Clin Invest, 118, 1979-1990 (2008); See also, Wiley et al. Immunity, 3, 673-682 (1995); Pan et al. Science, 276, 111-113 (1997); Sheridan et al. Science, 277, 818-821 (1997); Pan et al. Science, 277, 8150818 (1997); Walczak et al. EMBO J. 16, 5386-5397 (1997)], which is initiated upon the clustering of the receptors' intracellular death domains [Wang and El-Deiry (2003) Oncogene 22: 8628-8633; Kelley and Ashkenazi (2004) Current Opinion in Pharmacology 4: 333-339]. Subsequent trimerization of these receptors leads to the recruitment of the adaptor molecule FADD, the binding of pro-caspase-8 and -10, and the formation of the death-inducing signaling complex. Caspase-8 and -10 are subsequently cleaved allowing these now active zymogens to cleave and activate the effector caspases, caspase-3, -6, and -7. As a consequence, the cell is committed to apoptotic death.

The discovery of the TRAIL receptors and availability of their DNA and protein sequences [Pan et al., (1997) Science 276: 111-113; Pan et al., (1997) Science 277: 815-818; Sheridan et al., (1997) Science 277: 818-821; Degli-Esposti et al., (1997) Immunity 7: 821-830; Marsters et al., (1997) Current Biology 7: 1003-1006; Emery et al., (1997) Journal of Biological Chemistry 273: 14363-14367] have enabled the development of peptide agonists and antagonists that would be beneficial for the treatment of a number of diseases.

TRAIL and TRAIL receptor agonists are of interest for cancer therapy because they predominantly induce apoptosis in cancer cells, while sparing normal cells [Lawrence et al., (2001) Nature Medicine 7: 383-385]. Administration of TRAIL receptor agonists into a wide variety of experimental animal models of cancer induces significant tumor regression without systemic toxicity [reviewed in Ashkenazi and Herbst (2008) Journal of Clinical Investigation 6: 1979-1990; Kelley et al., (2001) Journal Pharmacol Exp Ther 299: 31-38; Ashkenazi et al., (1999) Journal of Clinical Investigation 104: 155-162; Walczak et al., (1999) Nature Medicine 5: 157-163]. Recent clinical studies have demonstrated that TRAIL receptor agonists are well tolerated by patients and deserve further clinical study [reviewed in Ashkenazi and Herbst (2008) Journal of Clinical Investigation 6: 1979-1990]. TRAIL receptor agonists have potential in the treatment of a wide range of malignancies that normally are treated with radiation or chemotherapy.

Targeting TRAIL receptors with peptide agonists is a useful therapeutic strategy to circumvent resistance to conventional approaches to treating cancer, such as radio- and chemotherapy. Unlike TRAIL or TRAIL peptide agonists, which utilize the p53-independent “extrinsic” pathway of apoptosis, conventional cancer therapies, such as those described above, utilize the “intrinsic” pathway to induce cell death, and therefore require intact p53 function. As tumors progress, or as a result of treatment with conventional therapies, p53 is mutated in over 50% of tumors, leading to resistance to conventional therapies [Hollstein et al., (1994) Nucleic Acids Research 22: 3551-3555; Sidransky and Hollstein (1996) Annual Review of Medicine 47:285-301; Lee and Bernstein (1995) Cancer Metastasis Review 14(2):149-161]. Thus, TRAIL peptide agonists would be a useful alternative treatment for these resistant tumors. Furthermore, in tumors that have retained the p53 response pathway, chemotherapy may induce increased TRAIL R2 expression via p53 activation [Wang and El-Deiry (2003) Proceedings of the National Academy of Sciences 100: 15095-15100; Nagane et al., (2001) Apoptosis 6: 191-197], and therefore, TRAIL receptor engagement may synergize with chemotherapy and radiation to enhance tumor cell apoptosis [Fulda (2008) Current Cancer Drug Targets 8(2):132-40].

TRAIL peptides may be useful as TRAIL R2 peptide antagonists for the treatment diseases such as asthma, which is a chronic airway disease triggered by exposure to a variety of stimuli such as allergens, environmental tobacco smoke, pet dander, moist air, exercise or exertion, or emotional stress. The currently available asthma drugs are anti-inflammatory and bronchodilator drugs that are effective for asthma control in many patients. However, a significant minority of patients have a more severe, persistent asthma which could benefit from new approaches to disease management, such as the use of TRAIL peptide antagonists. In fact, a role for TRAIL and TRAIL receptor signaling has been implicated in asthma. It has been shown that TRAIL expression within the airway epithelium initiates a complex immunological cascade typical of asthma, characterized by the influx of immune cells, such as eosinophils, mast cells, dendritic cells, and T cells, and resulting in the production of a large number of inflammatory mediators within the airways [Wills-Karp (1999) Annual Reviews of Immunology 17: 255-281; Kay et al., (2004) Trends in Immunology 25(9): 477-482; Rothenberg and Hogan (2006) Annual Review of Immunology 24: 147-174]. TRAIL may also contribute to the pathogenesis of asthma by prolonging the survival of eosinophils, a key cellular mediator of airway disease [Robertson et al., (2002) J. Immunol. 169: 5986-5996].

Recent studies suggest that TRAIL R2 is an important receptor in asthma that should be targeted in the next generation of therapeutic agents. Specifically, TRAIL has been identified as an early signal that is released from the respiratory epithelium in response to allergen exposure and promotes inflammation and bronchoconstriction in the airways. Both the expression of TRAIL and TRAIL receptors, including TRAIL R2, have been shown to be present in the airway of individuals with asthma following allergen provocation [Robertson et al., (2002) J. Immunol. 169: 5986-5996; Weckmann et al., (2007) Nature Medicine. 13(11):1308-1315], and TRAIL has been shown to play an essential role in promoting the pathogenesis of asthma [Weckmann et al., (2007) Nature Medicine. 13(11):1308-1315]. TRAIL gene disruption in the mouse abolishes airway hyperreactivity and reduces airway inflammation [Weckmann et al., (2007) Nature Medicine. 13(11):1308-1315], and silencing TRAIL expression in the lung using synthetic small interfering RNA molecules also abolishes allergic airway disease [Weckmann et al., (2007) Nature Medicine. 13(11):1308-1315].

TRAIL receptor activation can have both a positive impact (e.g., induction of apoptosis specifically in tumor cells) and a negative impact (e.g., the exacerbation of asthma). Thus, it is imperative that both agonistic (e.g., anti-cancer) and antagonistic (e.g., anti-asthma) therapeutic agents that can modulate TRAIL receptor signaling be developed. The present invention provides both peptide agonists and peptide antagonists that meet these needs.

### SUMMARY OF THE INVENTION

The present invention provides novel synthetic peptides and peptide-based compounds that are agonists of TRAIL R2 receptor. The present invention also provides novel synthetic peptides and peptide-based compounds that are antagonists of TRAIL R2 receptor. One embodiment of the current invention provides a compound comprising a peptide that binds to a TRAIL R2 receptor and comprises a sequence of amino acids Ac-W-D-C-L-D-N-X1-I-G-R-R-Q-C-V-X2-L-NH₂ (SEQ ID NO: 18), wherein each amino acid is indicated by standard one letter abbreviation, and wherein X1 and X2 are each independently selected from the amino acid residues arginine (R) or lysine (K).

Another embodiment of the current invention provides compound comprising a peptide that binds to a TRAIL R2 receptor and comprises a sequence of amino acids selected from the group consisting of:

| | |
|---|---|
| AcWDCLDNRIGRRQCVKL-NH2; | (SEQ ID NO: 19) |
| AcGGSWDCLDNRIGRRQCVKL-NH2; | (SEQ ID NO: 20) |
| AcWDCLDN(X3)IGRRQCVKL-NH2; | (SEQ ID NO: 21) |
| AcWDCLDRPGRRQCVK-NH2; | (SEQ ID NO: 22) |
| AcWDCLDNKIGRRQCVRL-NH2; | (SEQ ID NO: 23) |
| AcCLDNRIGRRQCV; | (SEQ ID NO: 24) |
| AcDCLDNRIGRRQCVKL-NH2; | (SEQ ID NO: 25) |
| AcWDCLDNRIGKRQCVRL-NH2; | (SEQ ID NO: 26) |
| AcWDCLDNRIG(X4)RQCV(X5)L-NH2; | (SEQ ID NO: 27) |
| AcWDCLDNRIGRRQCVK-NH2; | (SEQ ID NO: 28) |
| AcWDCLVDRPGRRQCVRLEK-NH2; | (SEQ ID NO: 29) |
| AcWDCLVDRPGRRQCVRLERK-NH2; | (SEQ ID NO: 30) |
| AcWDCLVDRPGRRQCVKLER-NH2; | (SEQ ID NO: 31) |
| GGGSWDCLDNRIGRRQCVKL; | (SEQ ID NO: 4) |
| AcCWDLDNRIGRRQVCKL-NH2; | (SEQ ID NO: 36) |
| and | |
| GGGSWDCLDNRIGRRQCVKL-NH2 | (SEQ ID NO: 32) |

wherein each amino acid is indicated by standard one letter abbreviation, and wherein X3, X4, and X5 are independently selected from the amino acid residues arginine (R) and lysine (K).

Another embodiment of the invention provides compound comprising a peptide that binds to a TRAIL R2 receptor and comprises a sequence of amino acids:

| | |
|---|---|
| AcWDCLDNRIGKRQCVR-NH2; | (SEQ ID NO: 33) |
| or | |
| AcWDCLDNRIGKRQCVRA-NH2. | (SEQ ID NO: 34) |

Other embodiments provide compounds comprising peptide sequences of the invention, wherein the peptide sequence is a monomer, dimer, homodimer, trimer, homotrimer, heterodimer, or heterotrimer. Other embodiments provide compounds comprising peptide sequences of the invention, wherein the peptide sequence is a peptide dimer based on peptide monomer sequences of the invention further comprising a linker. In some embodiments, the linker used with peptide sequences in compounds of the inventions is diglycolic acid (DIG) or Tris-succinimidyl aminotriacetate (TSAT). In other embodiments, compounds are provided that comprise peptide sequences of the invention, wherein the first amino acid residue of said peptide is acetylated.

In some embodiments, the invention provides a compound comprising a peptide trimer that binds to a TRAIL R2 receptor and where each peptide comprises a sequence of amino acids Ac-W-D-C-L-D-N-R-I-G-R-R-Q-C-V-K-L-NH₂ (SEQ ID NO: 19), wherein each amino acid is indicated by standard one letter abbreviation and AcW is N-acetyl-tryptophan.

Additional embodiments of the invention include a method for treating cancer in a patient, which method comprises administering to the patient a therapeutically effective amount of the compound comprising peptide sequences of the invention, wherein the peptide sequence is a monomer, dimer, homodimer, trimer, homotrimer, heterodimer, or heterotrimer. Other embodiments of the invention include a method for treating asthma in a patient, which method comprises administering to the patient a therapeutically effective amount of the compound comprising peptide sequences of the invention, wherein the peptide sequence is a monomer, dimer, homodimer, trimer, homotrimer, heterodimer, or heterotrimer. The present invention also includes a pharmaceutical composition comprising a compound of the invention and a pharmaceutically acceptable carrier.

Another embodiment of the invention provides a compound that binds to and activates a TRAIL R2 receptor, which compound comprises a peptide dimer of SEQ ID NO: 19 having the formula:

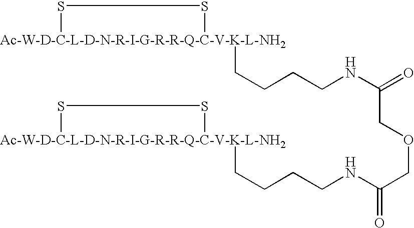

wherein (i) in each peptide monomer of the peptide dimer, each amino acid is indicated by standard one letter abbreviation and AcW is N-acetyl-tryptophan; and (ii) each peptide monomer of the peptide dimer contains an intramolecular disulfide bond between the two cysteine (C) residues of each peptide monomer.

Another embodiment of the invention provides a compound that binds to and activates a TRAIL R2 receptor, which compound comprises a peptide trimer of SEQ ID NO: 19 having the formula:

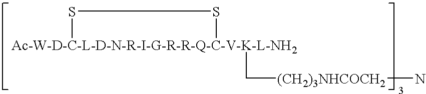

wherein (i) in each peptide monomer of the peptide trimer, each amino acid is indicated by standard one letter abbreviation and AcW is N-acetyl-tryptophan; and (ii) each peptide monomer of the peptide trimer contains an intramolecular disulfide bond between the two cysteine (C) residues of each peptide monomer.

Another embodiment of the invention provides a compound that binds to and antagonizes a TRAIL R2 receptor, which compound comprises a peptide trimer of SEQ ID NO: 34 having the formula:

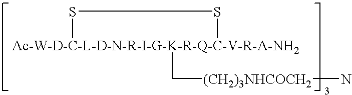

### BRIEF DESCRIPTION OF THE DRAWINGS

FIG. 1 is representative of results that are derived from raw data from an AlphaQuest® TRAIL R2 receptor binding competition assay.

FIG. 2 illustrates the TRAIL R2 hit-to-lead optimization strategies.

FIG. 3 is representative of pIC₅₀ (which is equivalent to −log₁₀ IC₅₀) values of truncated constructs of the hit peptide sequence that are derived from raw data from the AlphaQuest® TRAIL R2 receptor binding competition assay.

FIG. 4A is representative of results that are obtained from an alanine scan of the TRAIL R2 hit peptide sequence.

FIG. 4B is representative of binding activities that are obtained from raw data for peptide agonists of the invention using the AlphaQuest® TRAIL R2 receptor binding competition assay.

FIGS. 5A and 5B is representative of the optimization of a TRAIL agonist peptide sequence with apoptotic (i.e., functional) activity compared to a peptide that binds to TRAIL R2, but is without apoptotic activity.

FIG. 6 demonstrates that dimerization of the peptides of the invention increases binding activity compared to peptide monomers.

FIGS. 7A and 7B show the optimization of the linker position for the peptide homodimers of the invention.

FIGS. 8A and 8B illustrate examples of the trimerization of the peptides of the invention.

FIG. 9 shows a comparison of homodimers versus homotrimers in an HCT-116 proliferation assay.

FIG. 10 demonstrates the apoptotic activity of the peptide agonists of the invention in a Jurkat proliferation assay.

FIG. 11A illustrates a representative “TRAIL curve” for Jurkat cell apoptosis induced by TRAIL ligand using a Jurkat proliferation assay.

FIGS. 11B-D show that antagonist peptides of the invention inhibit the ability of TRAIL to induce apoptosis in Jurkat cells using the Jurkat antagonist assay. FIG. 11B shows the calculcated EC₅₀ value for the inhibition of TRAIL-ligand-induced apoptosis of Jurkat cells by a peptide homotrimer based upon the sequence of SEQ ID NO: 34. FIG. 11C shows the calculated EC₅₀ value for the inhibition of TRAIL-ligand-induced apoptosis of Jurkat cells by a peptide homodimer based upon the sequence of SEQ ID NO: 33. FIG. 11D shows the calculated EC₅₀ value for the inhibition of TRAIL-ligand-induced apoptosis of Jurkat cells by a peptide homodimer based upon the sequence of SEQ ID NO: 34.

### DETAILED DESCRIPTION OF THE INVENTION

The present invention provides peptides and peptide-based compounds that bind to a TRAIL receptor and function as agonists or, alternatively, as antagonists to the TRAIL receptor. In preferred embodiments, the peptides and peptide-based compounds of the invention bind to a TRAIL R2 receptor and further act as agonists or, alternatively, as antagonists of the TRAIL R2 receptor. Reference to TRAIL R2 receptor and TRAIL R2 are used throughout this application to refer to TRAIL R2 receptor. These compounds include “lead” peptide-based compounds and “derivative” compounds constructed so as to have the same or similar molecular structure or shape as the lead compounds, but that differ from the lead compounds, e.g., with respect to susceptibility to hydrolysis or proteolysis, and/or with respect to other biological properties, such as increased affinity for the receptor and/or functional activity. In certain embodiments, the present invention provides compositions comprising an effective amount of a TRAIL R2-binding, TRAIL R2-agonist compound, and more particularly a compound that is useful for treating cancer. In other embodiments, the present invention provides compositions having an effective amount of a TRAIL R2-binding, TRAIL R2 antagonist compound, and more particularly a compound that is useful for treating disorders (e.g., asthma) associated with the overexpression of TRAIL ligand, and/or with the production and accumulation of eosinophils.

The term “peptide” generally refers to a polypeptide (i.e., a polymer of amino acid residues joined together by an amide bond between adjacent amino acid residues) that is typically no more than a few dozen amino acids in length. In some embodiments, peptides are at least about 5, 6, 8, 10, 12, 14, 15, 16, 18, 19, 20, or 21 amino acid residues long. A polypeptide, in contrast with a peptide, may comprise any number of amino acid residues. Hence, the term polypeptide includes peptides as well as longer sequences of amino acids. The terms “polypeptide” and “peptide” encompass native or artificial proteins, protein fragments and polypeptide analogs of a protein sequence. Therefore, “peptides” of the invention are distinguishable over full-length proteins and other polypeptides, such as the full-length TRAIL protein and its receptors, each of which may be hundreds of amino acid residues in length.

The term “agonist” refers to a biologically active ligand, such as, but not limited to, synthetic agonists, which binds to its complementary biologically active receptor and activates the latter either to cause a biological response in the receptor, or to enhance preexisting biological activity of the receptor. The term “antagonist” refers to a peptide or peptide-based compound of the invention that binds to a receptor site of its cognate receptor, but does not retain the bioactivity of the native substrate of interest, or at least at a reduced level of activity relative to the native substrate, and inhibits the biological action of the native substrate. Agonists and antagonists may include peptides or peptide-based compounds of the invention as well as proteins, nucleic acids, carbohydrates, or any other molecules that associate with a peptide or peptide-based compound of the invention. One embodiment of the invention provides synthetic peptide agonists that bind to the TRAIL receptor. In another embodiment, the invention provides synthetic peptide agonists that bind to the TRAIL R2 receptor. One embodiment of the invention provides synthetic peptide antagonists that bind to the TRAIL receptor. In another embodiment, the invention provides synthetic peptide antagonists that bind to the TRAIL R2 receptor.

In one embodiment, the present invention provides a compound comprising a peptide that binds to TRAIL R2 and comprises a sequence of amino acids Ac-W-D-C-L-D-N-X1-1-G-R-R-Q-C-V-X2-L-NH₂ (SEQ ID NO: 18) wherein X1 is either R or K and X2 is either R or K. The amino acid sequences of exemplary peptide agonists of the invention are shown in Table 1, below, wherein X1, X2, X3, X4, and X5 are independently selected from the amino acids arginine (R) and lysine (K). The amino acid sequences of exemplary peptide antagonists of the invention are shown in Table 2, below.

TABLE 1

| SEQ ID | | | | | | | | | | | | | | | | | | | | |
|---|---|---|---|---|---|---|---|---|---|---|---|---|---|---|---|---|---|---|---|---|
| NO: | AGONIST AMINO ACID SEQUENCES | | | | | | | | | | | | | | | | | | | |
| 1 | W | D | C | L | D | N | X1 | I | G | R | R | Q | C | V | X2 | L | | | | |
| 2 | W | D | C | L | D | N | R | I | G | R | R | Q | C | V | K | L | | | | |
| 3 | G | G | S | W | D | C | L | D | N | R | I | G | R | R | Q | C | V | K | L | |
| 4 | G | G | G | S | W | D | C | L | D | N | R | I | G | R | R | Q | C | V | K | L |
| 5 | W | D | C | L | D | N | X3 | I | G | R | R | Q | C | V | K | L | | | | |
| 6 | W | D | C | L | D | R | P | G | R | R | Q | C | V | K | | | | | | |
| 7 | W | D | C | L | D | N | K | I | G | R | R | Q | C | V | R | L | | | | |
| 8 | C | L | D | N | R | I | G | R | R | Q | C | V | | | | | | | | |
| 9 | D | C | L | D | N | R | I | G | R | R | Q | C | V | K | L | | | | | |
| 10 | W | D | C | L | D | N | R | I | G | K | R | Q | C | V | R | L | | | | |
| 11 | W | D | C | L | D | N | R | I | G | X4 | R | Q | C | V | X5 | L | | | | |
| 12 | W | D | C | L | D | N | R | I | G | R | R | Q | C | V | K | | | | | |
| 13 | W | D | C | L | V | D | R | P | G | R | R | Q | C | V | R | L | E | K | | |
| 14 | W | D | C | L | V | D | R | P | G | R | R | Q | C | V | R | L | E | R | K | |
| 15 | W | D | C | L | V | D | R | P | G | R | R | Q | C | V | K | L | E | R | | |
| 35 | C | W | D | L | D | N | R | I | G | R | R | Q | V | C | K | L | | | | |

TABLE 2

| SEQ ID | | | | | | | | | | | | | | | | |
|---|---|---|---|---|---|---|---|---|---|---|---|---|---|---|---|---|
| NO: | ANTAGONIST AMINO ACID SEQUENCES | | | | | | | | | | | | | | | |
| 16 | W | D | C | L | D | N | R | I | G | K | R | Q | C | V | R | |
| 17 | W | D | C | L | D | N | R | I | G | K | R | Q | C | V | R | A |

Amino acid residues are abbreviated throughout the specification, using the standard single-letter and three-letter code routinely used in the biological art [See, e.g., Principles of Biochemistry, 2. Ed. (Lehninger, A. L., Nelson, D. L., & Cox, M. M.), New York, N.Y. (1993)]. In addition to any of the twenty “standard” naturally occurring amino acid residues, the peptides and peptide-based compounds of the invention may also comprise “non-standard” or “unconventional” amino acid residues. Examples of preferred unconventional amino acid residues in the peptides of the invention are: acetylated glycine (N-acetylglycine) (“AcG”); acetylated tryptophan (N-acetyl-tryptophan) (“AcW”); and acetylated-aspartic acid (N-acetyl-aspartic acid) (“AcD”). Additional examples of unconventional amino acid residues include, but are not limited to: β-alanine, 3-pyridylalanine, 4-hydroxyproline, O-phosphoserine, N-methylglycine, N-acetylserine, N-formylmethionine, 3-methylhistidine, 5-hydroxylysine, nor-leucine, and other similar amino acid and imino acid residues. In some embodiments, the peptides of the current invention are modified at the C-terminal end by the addition of an amino group (NH₂). In preferred embodiments of the invention, the amino acid residues of the peptide sequences may contain modified, unconventional amino acid residues. Preferred peptide agonist sequences of the invention are shown in Table 3, below, wherein X1, X2, X3, X4, and X5 are independently selected from the amino acids arginine (R) and lysine (K). Preferred peptide antagonist sequences of the invention are shown in Table 4, below.

TABLE 3

| SEQ ID | | | | | | | | | | | | | | | | | | | | | | |
|---|---|---|---|---|---|---|---|---|---|---|---|---|---|---|---|---|---|---|---|---|---|---|
| NO: | AGONIST AMINO ACID SEQUENCES | | | | | | | | | | | | | | | | | | | | | |
| 18 | Ac | W | D | C* | L | D | N | X1 | I | G | R | R | Q | C* | V | X2 | L | NH2 | | | | |
| 19 | Ac | W | D | C* | L | D | N | R | I | G | R | R | Q | C* | V | K | L | NH2 | | | | |
| 20 | Ac | G | G | S | W | D | C* | L | D | N | R | I | G | R | R | Q | C* | V | K | L | NH2 | |
| 21 | Ac | W | D | C* | L | D | N | X3 | I | G | R | R | Q | C* | V | K | L | NH2 | | | | |
| 22 | Ac | W | D | C* | L | D | R | P | G | R | R | Q | C* | V | K | NH2 | | | | | | |
| 23 | Ac | W | D | C* | L | D | N | K | I | G | R | R | Q | C* | V | R | L | NH2 | | | | |
| 24 | Ac | C* | L | D | N | R | I | G | R | R | Q | C* | V | | | | | | | | | |
| 25 | Ac | D | C* | L | D | N | R | I | G | R | R | Q | C* | V | K | L | NH2 | | | | | |
| 26 | Ac | W | D | C* | L | D | N | R | I | G | K | R | Q | C* | V | R | L | NH2 | | | | |
| 27 | Ac | W | D | C* | L | D | N | R | I | G | X4 | R | Q | C* | V | X5 | L | NH2 | | | | |
| 28 | Ac | W | D | C* | L | D | N | R | I | G | R | R | Q | C* | V | K | NH2 | | | | | |
| 29 | Ac | W | D | C* | L | V | D | R | P | G | R | R | Q | C* | V | R | L | E | K | NH2 | | |
| 30 | Ac | W | D | C* | L | V | D | R | P | G | R | R | Q | C* | V | R | L | E | R | K | NH2 | |
| 31 | Ac | W | D | C* | L | V | D | R | P | G | R | R | Q | C* | V | K | L | E | R | NH2 | | |
| 32 | | G | G | G | S | W | D | C* | L | D | N | R | I | G | R | R | Q | C* | V | K | L | NH2 |
| 36 | AC | C* | W | D | L | D | N | R | I | G | R | R | Q | V | C* | K | L | NH2 | | | | |

*= Cysteine residue of disulfide bond

TABLE 4

| SEQ ID | | | | | | | | | | | | | | | | | | |
|---|---|---|---|---|---|---|---|---|---|---|---|---|---|---|---|---|---|---|
| NO: | ANTAGONIST AMINO ACID SEQUENCES | | | | | | | | | | | | | | | | | |
| 33 | Ac | W | D | C* | L | D | N | R | I | G | K | R | Q | C* | V | R | NH2 | |
| 34 | Ac | W | D | C* | L | D | N | R | I | G | K | R | Q | C* | V | R | A | NH2 |

*= Cysteine residue of disulfide bond

Stereoisomers (e.g., D-amino acid residues) of the twenty conventional amino acid residues, unnatural amino acid residues such as α,α-disubstituted amino acid residues, N-alkyl amino acid residues, lactic acid, and other unconventional amino acid residues may also be suitable components for the peptides and peptide-based compounds of the present invention.

Peptides having substantial identity to the peptides and peptide-based compounds of the invention that retain activity similar to the peptides and peptide-based compounds of the invention are also included in the invention. As applied to peptides and polypeptides, the term “substantial identity” means that two peptide sequences, when optimally aligned share at least 70, 75 or 80 percent sequence identity, preferably at least 90 or 95 percent sequence identity, and more preferably at least 97, 98 or 99 percent sequence identity. The length of polypeptide sequences compared for homology will generally be at least about 16 amino acid residues, usually at least about 20 residues, more usually at least about 24 residues, typically at least about 28 residues, more typically at least about 35 residues, and preferably more than about 50 residues. When searching a database containing sequences from a large number of different organisms, it is preferable to compare amino acid sequences.

Protein analysis programs can be used to determine sequence identity, by matching similar sequences using measures of similarity assigned to various substitutions, deletions and other modifications, including conservative amino acid substitutions. For example, GCG software contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 11.0. Polypeptide sequences also can be compared using FASTA using default or recommended parameters, a program in GCG Version 11.0, FASTA (e.g., FASTA2 and FASTA3), provides alignments and percent sequence identity of the regions of the best overlap between the query and search sequences (Pearson, Methods Enzymol. 183:63-98 (1990); Pearson, Methods Mol. Biol. 132:185-219 (2000)). Another preferred algorithm when comparing a sequence of the invention to a database containing a large number of sequences from different organisms is the computer program BLAST, especially blastp or tblastn, using default parameters. See, e.g., Altschul et al., J. Mol. Biol. 215:403-410 (1990); Altschul et al., Nucleic Acids Res. 25:3389-402 (1997); herein incorporated by reference.

In some embodiments, the compound comprises a peptide that is a monomer, a peptide that is a dimer, a peptide that is a trimer, a peptide that is a tetramer, or a peptide that is a multimer. Preferably, the peptide multimer is a homomultimer; i.e., a multimer comprising a plurality of peptide monomers (e.g., two, three, four, or more peptide monomers) having the same amino acid sequences. However, the invention also includes heteromultimers that comprise a plurality of peptide monomers where two or more of the peptide monomers have different amino acid sequences.

Peptides of the invention may be multimerized via a linker. By “linker”, herein is meant a molecule or group of molecules (such as a monomer or polymer) that connects two molecules and often serves to place the two molecules in a preferred configuration. In one aspect of this embodiment, the linker is a peptide bond. Choosing a suitable linker for a specific case where two polypeptide chains are to be connected depends on various parameters, e.g., the nature of the two polypeptide chains (e.g., whether they naturally oligomerize (e.g., form a dimer or not), the distance between the N- and the C-termini to be connected if known from three-dimensional structure determination, and/or the stability of the linker towards proteolysis and oxidation. For example, in one embodiment, a lysine residue may be used. In other embodiments, other bi-functional linkers may be used. In addition, the compounds or peptides may contain cysteine residues for the purpose of introducing an intramolecular disulfide bridge or constraint at various locations in the amino acid sequence. Exemplary linker moieties are described in detail, infra, in this specification and its examples. A skilled artisan will be able to select appropriate linkers from both these and other linker moieties known in the art, as well as from other linkers that may be subsequently developed. In particular, the skilled artisan will recognize that the substitution of a particular linker moiety may be useful for optimizing binding and/or other functional properties.

The term “intramolecular bond” refers to a chemical bond between two or more atoms in the single molecule, such as, for example, a chemical bond between two functional groups of a single molecule. The term “intermolecular bond” refers to a chemical bond between two or more atoms that form different molecules. Typically, an intramolecular bond includes one or more covalent bonds, such as, for example, σ-bonds, π-bonds, and coordination bonds. The term “conjugated π-bond” refers to a π-bond that has a π-orbital overlapping (e.g., substantially overlapping) a π-orbital of an adjacent 1-bond. Additional examples of bonds include various mechanical, physical, and electrical couplings. The term “bond” and its grammatical variations refer to a coupling or joining of two or more chemical or physical elements. In some instances, a bond can refer to a coupling of two or more atoms based on an attractive interaction, such that these atoms can form a stable structure. Examples of bonds include chemical bonds such as chemisorptive bonds, covalent bonds, ionic bonds, van der Waals bonds, and hydrogen bonds.

The term “group” as applies to chemical species refers to a set of atoms that forms a portion of a molecule. In some instances, a group can include two or more atoms that are bonded to one another to form a portion of a molecule. A group can be monovalent or polyvalent (e.g., bivalent) to allow bonding to one or more additional groups of a molecule. For example, a monovalent group can be envisioned as a molecule with one of its hydrogen atoms removed to allow bonding to another group of a molecule. A group can be positively or negatively charged. For example, a positively charged group can be envisioned as a neutral group with one or more protons (i.e., H+) added, and a negatively charged group can be envisioned as a neutral group with one or more protons removed. Examples of groups include, but are not limited to, alkyl groups, alkylene groups, alkenyl groups, alkenylene groups, alkynyl groups, alkynylene groups, aryl groups, arylene groups, iminyl groups, iminylene groups, hydride groups, halo groups, hydroxy groups, alkoxy groups, carboxy groups, thio groups, alkylthio groups, disulfide groups, cyano groups, nitro groups, amino groups, alkylamino groups, dialkylamino groups, silyl groups, and siloxy groups.

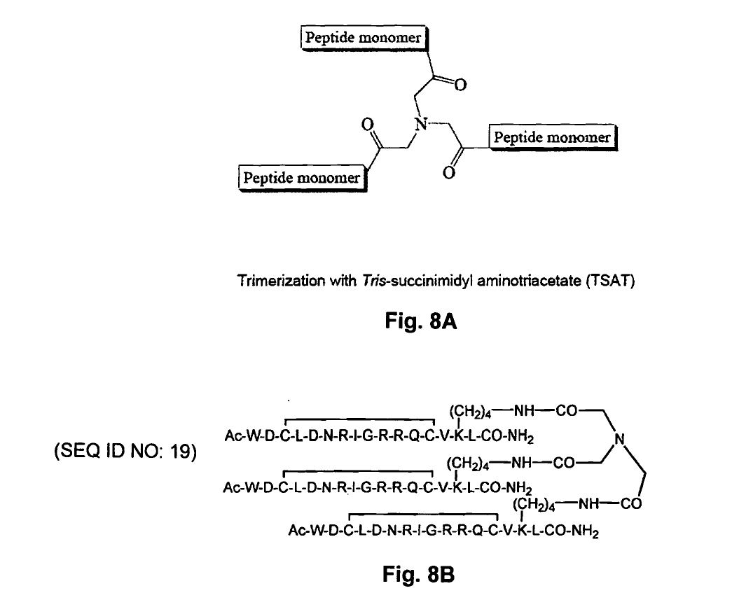

In one embodiment, the present invention provides a compound comprising a peptide homotrimer that binds to the TRAIL R2 receptor and comprises a sequence of amino acids Ac-W-D-C-L-D-N-R-I-G-R-R-Q-C-V-K-L-NH₂ (SEQ ID NO: 19) where each amino acid is indicated by standard one letter abbreviation (See, e.g., FIG. 8B).

In some embodiments, the agonist peptides or agonist peptide-based compounds may trigger tumor cell apoptosis upon binding to a TRAIL R2 receptor.

In other embodiments, the antagonist peptides or antagonist peptide-based compounds may inhibit TRAIL-induced inflammation in asthma or other, conditions that may benefit from antagonist activity of a TRAIL R2 binding peptide.

In all embodiments, the peptides or peptide-based compounds may contain an intramolecular disulfide bond between the cysteine residues of each monomer. Such monomers may be represented schematically as exemplified by the following structure:

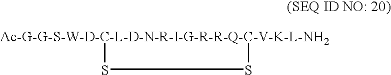

In certain embodiments, disulfide bonds may be used to generate cyclized peptides of different ring sizes by changing the positions of the cysteine residues of the peptide monomer. In the example below, the upper peptide homodimer based on the sequence of peptide monomer of SEQ ID NO: 19 has cysteine residues at position 3 and 13 of each peptide monomer, wherein 9 amino acids are within the cysteine loop; and in the lower peptide homodimer based on the sequence of peptide monomer of SEQ ID NO: 36, the cysteine residues are positioned at position 1 and 14 of each peptide monomer, wherein 12 amino acids are contained within the cysteine loop.

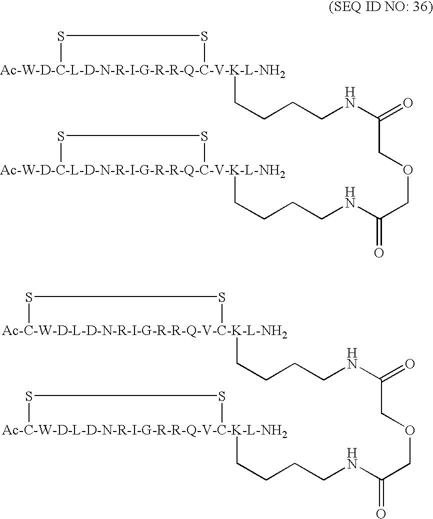

Amino Acid Substitutions of Peptides and Peptide-Based Compounds of the Invention

The amino acid sequences of peptides and the peptide-based compounds of the invention may be substituted. In some embodiments, the amino acid substitutions may be conservative or non-conservative. In other embodiments, peptides and peptide-based compounds of the invention may also comprise an amino acid sequence as set forth herein, but having one or more amino acid substitutions, additions, insertions, or deletions. In yet another embodiment, peptides and peptide-based compounds of the invention may also contain truncations, inversions, and rearrangement of the order of the amino acid residues. Preferably, amino acid residue positions that are not identical differ by conservative amino acid substitutions. A “conservative amino acid substitution” is one in which an amino acid residue is substituted by another amino acid residue having a side chain R group with similar chemical properties (e.g., charge or hydrophobicity). Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

In general, a conservative amino acid substitution will not substantially change the functional properties of a protein or the structural characteristics of the parent sequence (e.g., a replacement amino acid should not tend to break a helix that occurs in the parent sequence, or disrupt other types of secondary or tertiary structure that characterizes the parent sequence). Examples of art-recognized polypeptide secondary and tertiary structures are described in Proteins, Structures and Molecular Principles (Creighton, Ed., W.H. Freeman and Company, New York (1984)); Introduction to Protein Structure (C. Branden and J. Tooze, eds., Garland Publishing, New York, N.Y. (1991)); and Thornton et al., Nature 354:105 (1991)]. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. [See, e.g., Pearson, Methods Mol. Biol. 243:307-31 (1994).] Additionally, a conservative replacement is any change having a positive value in the PAM250 log-likelihood matrix disclosed in Gonnet et al., Science 256:1443-45 (1992). A “moderately conservative” replacement is any change having a nonnegative value in the PAM250 log-likelihood matrix.

Other preferred amino acid substitutions are those which: (1) reduce susceptibility to proteolysis, (2) reduce susceptibility to oxidation, (3) alter binding affinity for forming protein complexes, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts).

Modifications of Peptides and Peptide-Based Compounds of the Invention

Peptides and peptide-based compounds of the invention may be modified, and may be used to produce other compounds of the invention. These modifications include but are not limited to modification of the amino terminus (e.g. the amino terminus is acetylated with acetic acid or a halogenated derivative thereof such as α-chloroacetic acid, α-bromoacetic acid, or α-iodoacetic acid), modification of the carboxy terminus, and/or modification of the side chain of one or more amino acid residues, including, for example, phosphorylation, prenylation, acylation, O- and N-glycosylation, nucleosidylation, vitamin K-dependent carboxylation, hydroxylation, crosslinking, disulfide formation, methylation, ring substitution, disulfide reduction and/or oxidation.

One can replace the naturally occurring side chains of the 20 genetically encoded amino acid residues (or the stereoisomeric D amino acid residues) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclic. In particular, proline analogues in which the ring size of the proline residue may be changed from 5 members to 4, 6, or 7 members can be employed. Cyclic groups can be saturated or unsaturated, and if unsaturated, can be aromatic or non-aromatic. Heterocyclic groups preferably contain one or more nitrogen, oxygen, and/or sulfur heteroatoms. Examples of such groups include the furazanyl, furyl, imidazolidinyl, imidazolyl, imidazolinyl, isothiazolyl, isoxazolyl, morpholinyl (e.g., morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl. These heterocyclic groups can be substituted or unsubstituted. Where a group is substituted, the substituent can be alkyl, alkoxy, halogen, oxygen, or substituted or unsubstituted phenyl.

The peptides and peptide-based compounds of the invention also serve as structural models for non-peptidic compounds with similar biological activity. Those of skill in the art recognize that a variety of techniques are available for constructing compounds with the same or similar desired biological activity as the lead peptide or peptide-based compound, but with more favorable activity than the lead with respect to solubility, stability, and susceptibility to hydrolysis and proteolysis [See, Morgan and Gainor (1989) Ann. Rep. Med. Chem. 24:243-252].

The peptides and peptide-based compounds of the invention can also be expressed as or attached to a fusion protein, derivatized, labeled, or linked to another molecule (e.g., another peptide or protein, a small molecule, ligand, or a peptide analogue). As used herein, the terms “label” or “labeled” refers to incorporation of another molecule in the peptide or peptide-based compound. The peptides and peptide-based compounds of the invention may also be labeled with a predetermined polypeptide epitope recognized by a secondary reporter such as, for example, leucine zipper pair sequences, binding sites for secondary antibodies, metal binding domains, epitope tags, fluorescent labels (e.g., FITC, rhodamine, lanthanide phosphors), enzymatic labels (e.g., horseradish peroxidase, β-galactosidase, luciferase, alkaline phosphatase), and chemiluminescent markers, biotinyl groups, magnetic agents (e.g., gadolinium chelates). In some embodiments, labels are attached by spacer arms of various lengths to reduce potential steric hindrance.

In general, the peptides and peptide-based compounds are derivatized such that binding of the peptides and peptide-based compounds is not affected adversely by the derivatization or labeling. Accordingly, the peptides and peptide-based compounds of the invention are intended to include both intact and modified forms of the peptides and peptide-based compounds described herein. For example, a peptide or peptide-based compound of the invention can be functionally linked (by chemical coupling, genetic fusion, noncovalent association or otherwise) to one or more other molecular entities, such as to an antibody (e.g., a bispecific antibody or a diabody), a chemotherapeutic agent, a pharmaceutical agent, an anti-inflammatory agent and/or a protein or peptide that can mediate association of the peptide or peptide-based compound with another molecule (such as a streptavidin core region or a polyhistidine tag).

In one embodiment, the peptides and peptide-based compounds of the invention may be derivatized with a detecting agent. Useful detection agents, with which a peptide or peptide-based compound of the invention may be derivatized, include but are not limited to fluorescent compounds such as fluorescein, fluorescein isothiocyanate, rhodamine, 5-dimethylamine-1-napthalenesulfonyl chloride, phycoerythrin, and lanthanide phosphors. Peptides and peptide-based compounds of the invention may be labeled with enzymes that are useful for detection, such as, but not limited to, horseradish peroxidase, β-galactosidase, luciferase, alkaline phosphatase, and glucose oxidase. When a peptide or peptide-based compound of the invention is labeled with a detectable enzyme, it is detected by adding additional reagents that the enzyme uses to produce a reaction product that can be discerned. For example, when the agent horseradish peroxidase is present, the addition of hydrogen peroxide and diaminobenzidine leads to a colored reaction product, which is detectable.

In another embodiment, a derivatized peptide or peptide-based compound of the invention is produced by crosslinking two or more peptides (of the same peptide or of different types, e.g.; to create homodimeric, heterodimeric, heteromultimeric, or homomultimeric peptides and peptide-based compounds). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company (Rockford, Ill.). Additional examples include diglycolic acid (DIG) [See, e.g., U.S. Patent Application No. 2006-0014680 to Xu, et al.] and the amine-reactive trifunctional cross-linking agent, aminotriacetate (ATA) (also known as aminotriacetic acid) or its activated version, Tris-succinimidyl aminotriacetate (TSAT) [See, e.g, U.S. Patent Application No. 2005-0221316 A1, to Pedersen et al.].

The peptides and peptide-based compounds of the invention can also be labeled with a radiolabeled amino acid. The radiolabel can be used for both diagnostic and therapeutic purposes. For instance, the radiolabel can be used to detect TRAIL R2-expressing cells in vivo by x-ray or other diagnostic techniques. Further, radiolabeled peptide-agonists can be used therapeutically as a chemotherapeutic agent to induce apoptosis in tumors. Radiolabeled peptide antagonists of the invention can be used therapeutically as an anti-inflammatory agent to prevent or treat asthma. Examples of labels for peptides and peptide-based compounds of the invention include, but are not limited to, the following radioisotopes or radionuclides: ³H, ¹⁴C, ¹⁵N, ³⁵S, ⁹⁰Y, ⁹⁹Tc, ¹¹¹In, ¹²⁵I, and ¹³¹L.

In another aspect, the present invention provides medicaments and methods of using the same for the treatment of disease(s) in a subject. In one embodiment, the diseases suitable for treatment include cancer. In other embodiments, the diseases suitable for treatment include asthma. In yet another embodiment, the invention provides methods of treating the conditions described herein comprising administering the compounds or peptides described herein in a pharmaceutically acceptable form. In all embodiments, the subject is a mammalian subject, preferably human.

In one other aspect, the present invention provides methods of making the peptides or compounds described herein.

Peptides and Peptide-Based Compounds of the Invention

The peptide monomers of this invention may be dimerized or trimerized to provide peptide dimers and trimers with enhanced functional activity. In one embodiment, the linker moiety may be, for example, a lysine residue, which bridges the C-termini of two peptide monomers, by simultaneous attachment to the C-terminal amino acid residue of each peptide monomer. One peptide monomer is attached at its C-terminus to the lysine's ε-amino group and the second peptide monomer is attached at its C-terminus to the lysine's α-amino group. In other embodiments, the linker moiety is diglycolic acid (DIG) [See, e.g., U.S. Patent Application No. 2006-0014680 to Xu, et al.] or the amine-reactive trifunctional cross-linking agent, aminotriacetate (ATA) or its activated version, Tris-succinimidyl aminotriacetate (TSAT) [See, e.g, U.S. Patent Application No. 2005-0221316 A1, to Pedersen et al.], as shown below:

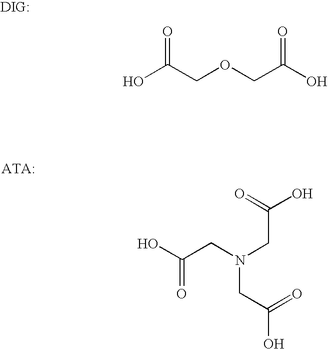

Examples of peptide monomers, dimers, trimers, and tetramers of the present invention may be represented schematically as follows:

1. A peptide homodimer based on the peptide monomer of SEQ ID NO: 19:

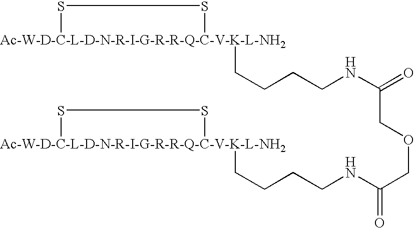

2. A peptide homodimer based on the peptide monomer of SEQ ID NO: 28:

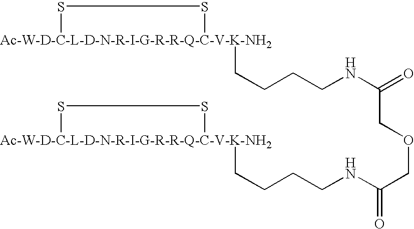

3. A peptide homodimer based on the peptide monomer of SEQ ID NO: 26:

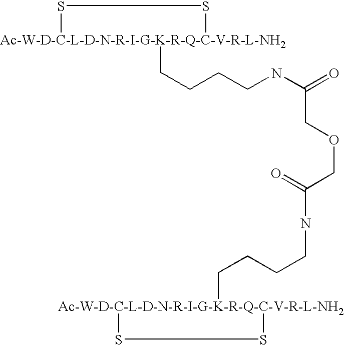

4. A peptide homodimer based on the peptide monomer of SEQ ID NO: 33:

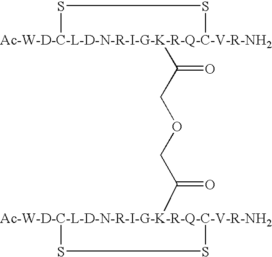

5. A peptide homodimer based on the peptide monomer of SEQ ID NO: 34:

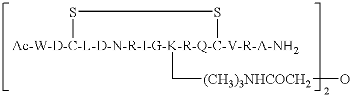

6. A peptide homodimer based on the peptide monomer of SEQ ID NO: 31:

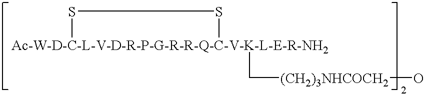

7. A peptide homodimer based on the peptide monomer of SEQ ID NO: 29:

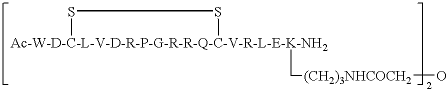

8. A peptide homodimer based on the peptide monomer of SEQ ID NO: 22:

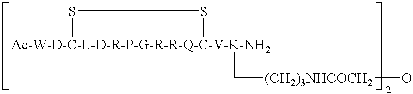

9. A peptide homodimer based on the peptide monomer of SEQ ID NO: 19:

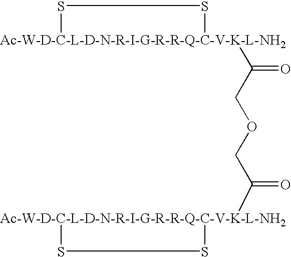

10. A peptide homotrimer based on the peptide monomer of SEQ ID NO: 20:

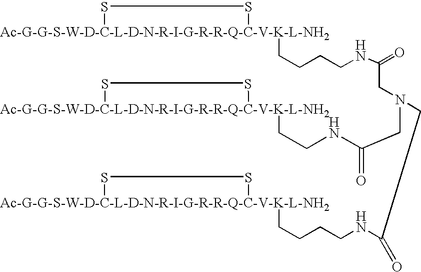

11. A peptide homotrimer based on the peptide monomer of SEQ ID NO: 19:

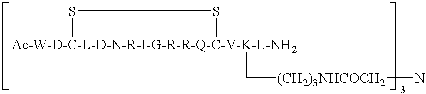

12. A peptide homotrimer based on the peptide monomer of SEQ ID NO: 34:

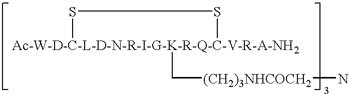

13. A peptide homotetramer based on the peptide monomer of SEQ ID NO: 19:

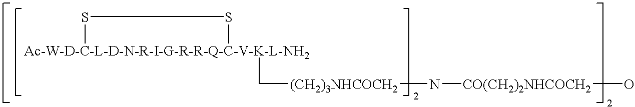

Preparation of the Peptides and Peptide-Based Compounds of the Invention

Synthesis

The peptide sequences of the present invention may be present alone or in conjunction with N-terminal and/or C-terminal extensions of the peptide chain. Such extensions may be naturally encoded peptide sequences optionally with or substantially without non-naturally occurring sequences; the extensions may include any additions, deletions, point mutations, or other sequence modifications or combinations as desired by those skilled in the art. For example and not limitation, naturally-occurring sequences may be full-length or partial length and may include amino acid residue substitutions to provide a site for attachment of carbohydrate, PEG, other polymer, or the like via side chain conjugation. In a variation, the amino acid residue substitution results in humanization of a sequence to make in compatible with the human immune system. Fusion proteins of all types are provided, including immunoglobulin sequences adjacent to or in near proximity to the sequences of the agonist and antagonist peptides of the present invention with or without a non-immunoglobulin spacer sequence. One exemplary embodiment is an immunoglobulin chain having the sequences of the agonist or antagonist peptides of the invention in place of the variable (V) region of the heavy and/or light chain.

The peptides of the invention may be prepared by classical methods known in the art. These standard methods include exclusive solid phase synthesis, partial solid phase synthesis methods, fragment condensation, classical solution synthesis, and recombinant DNA technology [See, e.g., Merrifield J. Am. Chem. Soc. 1963 85:2149].

A preferred method for peptide synthesis is solid phase synthesis. Solid phase peptide synthesis procedures are well-known in the art [see, e.g., Stewart Solid Phase Peptide Syntheses (Freeman and Co.: San Francisco) 1969; 2002/2003 General Catalog from Novabiochem Corp, San Diego, USA; Goodman Synthesis of Peptides and Peptidomimetics (Houben-Weyl, Stuttgart) 2002]. In solid phase synthesis, synthesis is typically commenced from the C-terminal end of the peptide using an α-amino protected resin. A suitable starting material can be prepared, for instance, by attaching the required α-amino acid residue to a chloromethylated resin, a hydroxymethyl resin, a polystyrene resin, a benzhydrylamine resin, or the like. One such chloromethylated resin is sold under the trade name BIO-BEADS SX-1 by Bio Rad Laboratories (Richmond, Calif.). The preparation of the hydroxymethyl resin has been described [Bodonszky, et al. (1966) Chem. Ind. London 38:1597]. The benzhydrylamine (BHA) resin has been described [Pietta and Marshall (1970) Chem. Commun. 650], and the hydrochloride form is commercially available from Beckman Instruments, Inc. (Palo Alto, Calif.). For example, an α-amino protected amino acid residue may be coupled to a chloromethylated resin with the aid of a cesium bicarbonate catalyst, according to the method described by Gisin (1973) Helv. Chim. Acta 56:1467.

After initial coupling, the α-amino protecting group is removed, for example, using solutions in organic solvents at room temperature. Thereafter, α-amino protected amino acid residues are successively coupled to a growing support-bound peptide chain. The α-amino protecting groups are those known to be useful in the art of stepwise synthesis of peptides, including: acyl-type protecting groups (e.g., formyl, trifluoroacetyl, acetyl), aromatic urethane-type protecting groups [e.g., benzyloxycarboyl (Cbz) and substituted Cbz], aliphatic urethane protecting groups [e.g., t-butyloxycarbonyl (Boc), isopropyloxycarbonyl, cyclohexyloxycarbonyl], and alkyl type protecting groups (e.g., benzyl, triphenylmethyl), fluorenylmethyl oxycarbonyl (Fmoc), allyloxycarbonyl (Alloc), and 1-(4,4-dimethyl-2,6-dioxocyclohex-1-ylidene)ethyl (Dde).

The side chain protecting groups (typically ethers, esters, trityl, PMC, and the like) remain intact during coupling and is not split off during the deprotection of the amino-terminus protecting group or during coupling. The side chain protecting group must be removable upon the completion of the synthesis of the final peptide and under reaction conditions that will not alter the target peptide. The side chain protecting groups for Tyr include tetrahydropyranyl, tert-butyl, trityl, benzyl, Cbz, Z-Br-Cbz, and 2,5-dichlorobenzyl. The side chain protecting groups for Asp include benzyl, 2,6-dichlorobenzyl, methyl, ethyl, and cyclohexyl. The side chain protecting groups for Thr and Ser include acetyl, benzoyl, trityl, tetrahydropyranyl, benzyl, 2,6-dichlorobenzyl, and Cbz. The side chain protecting groups for Arg include nitro, Tosyl (Tos), Cbz, adamantyloxycarbonyl mesitylsulfonyl (Mts), 2,2,4,6,7-pentamethyldihydrobenzofurane-5-sulfonyl (Pbf), 4-methoxy-2,3,6-trimethyl-benzenesulfonyl (Mtr), or Boc. The side chain protecting groups for Lys include Cbz, 2-chlorobenzyloxycarbonyl (2-Cl-Cbz), 2-bromobenzyloxycarbonyl (2-Br-Cbz), Tos, or Boc.

After removal of the α-amino protecting group, the remaining protected amino acid residues are coupled stepwise in the desired order. Each protected amino acid residue is generally reacted in about a 3-fold excess using an appropriate carboxyl group activator such as 2-(1H-benzotriazol-1-yl)-1,1,3,3 tetramethyluronium hexafluorophosphate (HBTU) or dicyclohexylcarbodiimide (DCC) in solution, for example, in methylene chloride (CH₂Cl₂), N-methyl pyrrolidone, dimethyl formamide (DMF), or mixtures thereof.

After the desired amino acid sequence has been completed, the desired peptide is decoupled from the resin support by treatment with a reagent, such as trifluoroacetic acid (TFA) or hydrogen fluoride (HF), which not only cleaves the peptide from the resin, but also cleaves all remaining side chain protecting groups. When a chloromethylated resin is used, hydrogen fluoride treatment results in the formation of the free peptide acids. When the benzhydrylamine resin is used, hydrogen fluoride treatment results directly in the free peptide amide. Alternatively, when the chloromethylated resin is employed, the side chain protected peptide can be decoupled by treatment of the peptide resin with ammonia to give the desired side chain protected amide or with an alkylamine to give a side chain protected alkylamide or dialkylamide. Side chain protection is then removed in the usual fashion by treatment with hydrogen fluoride to give the free amides, alkylamides, or dialkylamides.

In preparing the esters of the invention, the resins used to prepare the peptide acids are employed, and the side chain protected peptide is cleaved with base and the appropriate alcohol (e.g., methanol). Side chain protecting groups are then removed in the usual fashion by treatment with hydrogen fluoride to obtain the desired ester.

These procedures can also be used to synthesize peptides in which amino acid residues other than the 20 naturally occurring, genetically encoded amino acid residues or synthetic amino acid residues (e.g., N-methyl, L-hydroxypropyl, L-3,4-dihydrooxyphenylalanyl, δ amino acid residues such as L-δ-hydroxylysyl and D-δ-methylalanyl, L-α-methylalanyl, β amino acid residues, and isoquinolyl) are substituted as discussed in the foregoing disclosure, at one, two, or more positions of any of the compounds of the invention. D-amino acid residues and non-naturally occurring synthetic amino acid residues can also be incorporated into the peptides of the present invention. Examples of such procedures are described in U.S. Pat. No. 7,084,245 to Holmes, et al., and in U.S. Patent Application Nos US 2005-0107297 A1 to Holmes et al., US 2007-0027074 A1 to Holmes, et al., and US 2007-0032408 A1 to Holmes, et al.

Preparation of Peptide Dimers

In one embodiment, the peptide monomers of a peptide dimer are synthesized individually and dimerized subsequent to synthesis. For example, two peptide monomers of the invention are dimerized by a lysine linker moiety after peptide synthesis. Such conjugation may be achieved by methods well established in the art. In one embodiment, the linker contains at least two functional groups suitable for attachment to the target functional groups of the synthesized peptide monomers. For example, the lysine's two free amine groups may be reacted with the C-terminal carboxyl groups of each of two peptide monomers. The peptide monomers of the invention may be dimerized using a bifunctional linker, such as, but not limited to DIG or a lysine residue. Examples of suitable linkers are such as those described in U.S. Pat. No. 7,084,245 to Holmes, et al.; U.S. Publication Nos. US 2005-0107297 A1 to Holmes, et al.; US-2007-0032408 A1 to Holmes, et al.; U.S. Patent Application No. 2006-0014680 to Xu, et al; and .S. Patent Application No. 2005-0221316 A1, to Pedersen et al.

In still another embodiment, the peptide monomers of a dimer are linked via their C-termini by a branched tertiary amide linker moiety having two functional groups capable of serving as initiation sites for peptide synthesis and a third functional group (e.g., a carboxyl group or an amino group) that enables binding to another molecular moiety (e.g., as may be present on the surface of a solid support). In this case, the two peptide monomers may be synthesized directly onto two reactive nitrogen groups of the linker moiety in a variation of the solid phase synthesis technique. Such synthesis may be sequential or simultaneous.

An example of the synthesis of a peptide homodimer with SEQ ID NO: 20 using the bifunctional linker N-hydroxy succinimide (NHS)-activated DIG, is shown below:

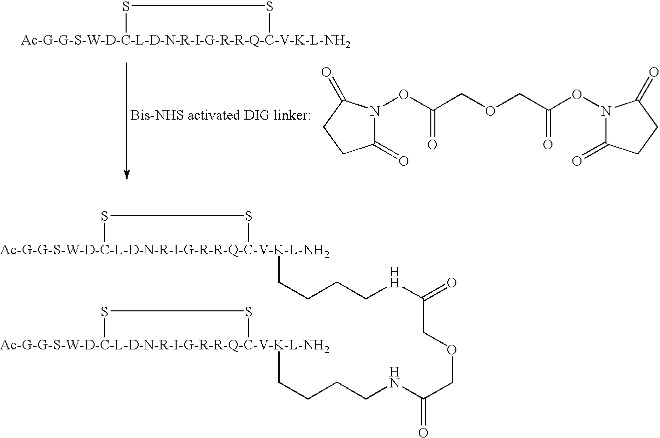

In other embodiments, the two peptide monomers may be synthesized directly onto two reactive nitrogen groups of the linker moiety in a variation of the solid phase synthesis technique. Such synthesis may be sequential or simultaneous. For example, a lysine linker moiety having two amino groups capable of serving as initiation sites for peptide synthesis and a third functional group (e.g., the carboxyl group of a lysine; or the amino group of a lysine amide, a lysine residue wherein the carboxyl group has been converted to an amide moiety —CO—NH₂) that enables binding to another molecular moiety (e.g., as may be present on the surface of a solid support) is used. In one embodiment, the lysine linker is incorporated into the peptide during peptide synthesis. For example, where a lysine linker moiety contains two functional groups capable of serving as initiation sites for peptide synthesis and a third functional group (e.g., a carboxyl group or an amino group) that enables binding to another molecular moiety, the linker may be conjugated to a solid support. Thereafter, two peptide monomers may be synthesized directly onto the two reactive nitrogen groups of the lysine linker moiety in a variation of the solid phase synthesis technique.

Where sequential synthesis of the peptide chains of a dimer onto a linker is to be performed, two amine functional groups on the linker molecule are protected with two different orthogonally removable amine protecting groups. The protected linker is coupled to a solid support via the linker's third functional group. The first amine protecting group is removed, and the first peptide of the dimer is synthesized on the first deprotected amine moiety. Then the second amine protecting group is removed, and the second peptide of the dimer is synthesized on the second deprotected amine moiety. For example, the first amino moiety of the linker may be protected with Alloc, and the second with Fmoc. In this case, the Fmoc group (but not the Alloc group) may be removed by treatment with a mild base [e.g., 20% piperidine in dimethyl formamide (DMF)], and the first peptide chain synthesized. Thereafter the Alloc group may be removed with a suitable reagent [e.g., Pd(PPh₃)/4-methyl morpholine and chloroform], and the second peptide chain synthesized. Note that where different thiol-protecting groups for cysteine are to be used to control disulfide bond formation (as discussed below) this technique must be used even where the final amino acid sequences of the peptide chains of a dimer are identical.

Where simultaneous synthesis of the peptide chains of a dimer onto a linker is to be performed, two amine functional groups of the linker molecule are protected with the same removable amine protecting group. The protected linker is coupled to a solid support via the linker's third functional group. In this case the two protected functional groups of the linker molecule are simultaneously deprotected, and the two peptide chains simultaneously synthesized on the deprotected amines. Note that using this technique, the sequences of the peptide chains of the dimer will be identical, and the thiol-protecting groups for the cysteine residues are all the same.

Preparation of Peptide Trimers and Tetramers

Preferred compounds of the invention include peptide trimers. Peptide trimers may be heterotrimers (i.e., consist of three unique peptide sequences or, alternatively two of the same peptides and one unique peptide). Peptide monomers of the invention may be combined with other peptides to form the heterotrimers. Preferred trimers are homotrimers (i.e., consist of three identical peptide monomers). In alternative embodiments, compounds of the invention may be tetramers. Tetramers may be synthesized by combining peptide dimers of the invention. Different combinations of peptide dimers to form distinct peptide tetramers are possible. Tetramers may also be prepared using peptide monomers of the invention.

In some embodiments, homotrimers of peptides of the invention may be prepared using trifunctional PEG.

A homotrimer can be formed by treating the peptide monomer sequence of SEQ ID NO: 20 with a trifunctional linker, such as the Tris-succinimidyl aminotriacetate linker shown below.

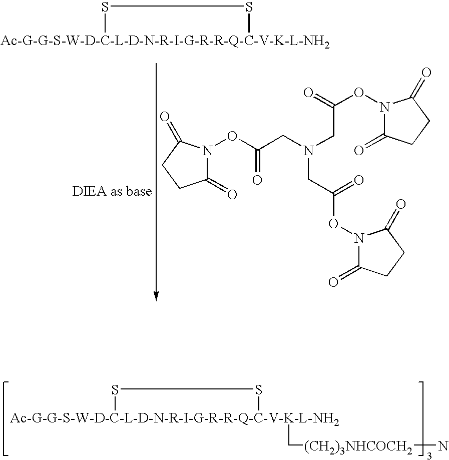

In another embodiment of the invention, a method to prepare a homotrimer is to first prepare a peptide homodimer using the sequence of SEQ ID NO: 20 with a functional group capable of forming a covalent bond with another peptide. For example, treating a peptide monomer, for example having the sequence of SEQ ID NO: 24, with the bis-activated iminodiacetic acid linker (IDA) affords an intermediate dimer species that can be further conjugated with an additional peptide of the same or different sequence, after removal of the Boc protecting group, as shown below:

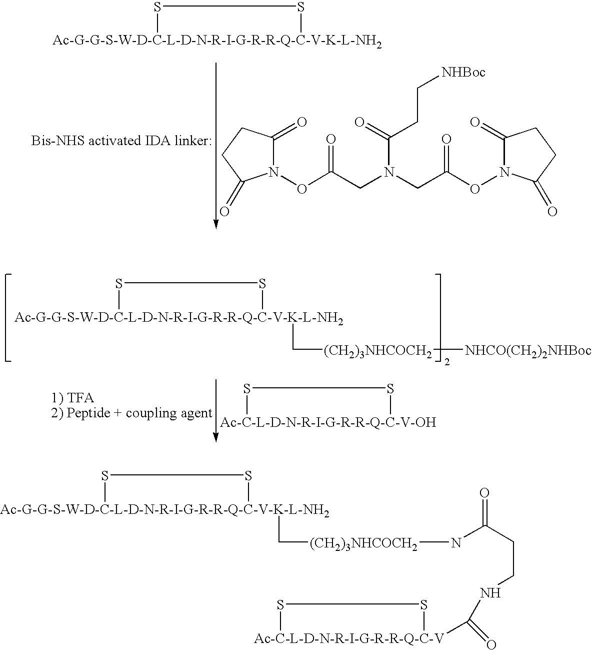

In another embodiment, a tetramer can be prepared by treating a peptide homodimer of the sequence of SEQ ID NO: 20 containing a reactive group capable of forming a covalent bond with a bifunctional linker, as shown below:

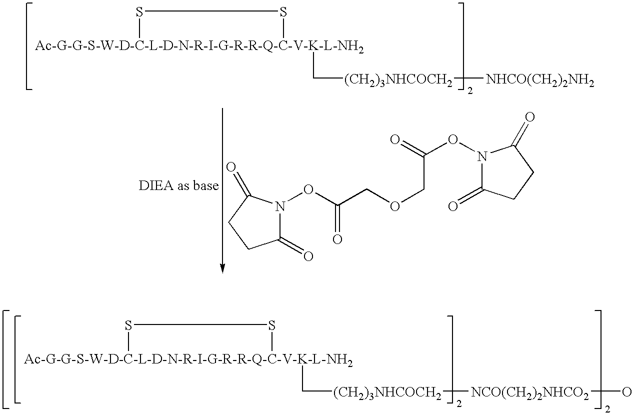

In another embodiment, the synthesis of a peptide tetramer based on the sequence of SEQ ID NO: 20 is shown below:

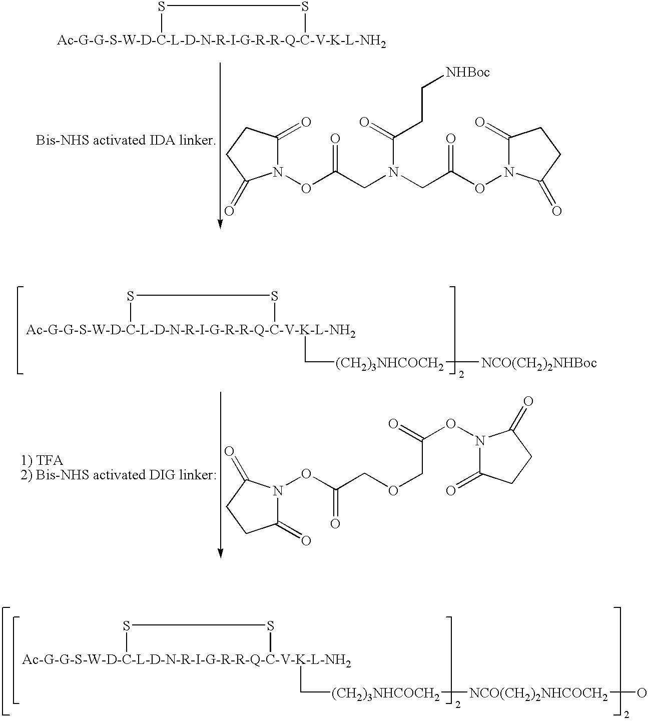

In another embodiment of the invention, a method of preparing a tetramer is to treat a peptide containing a functional group capable of forming a covalent bond with a tetrafunctional linker.

Formation of Disulfide Bonds

The peptides and peptide-based compounds of the present invention may contain one or more intramolecular disulfide bonds. Such disulfide bonds may be formed by oxidation of the cysteine residues of each peptide monomer.

In one embodiment, the control of cysteine bond formation is exercised by choosing an oxidizing agent of the type and concentration effective to optimize formation of the desired isomer. For example, oxidation of a peptide dimer to form two intramolecular disulfide bonds (one on each peptide chain) is preferentially achieved (over formation of intermolecular disulfide bonds) when the oxidizing agent is dimethyl sulfoxide (DMSO) or iodine (I₂).

In other embodiments, the formation of cysteine bonds is controlled by the selective use of thiol-protecting groups during peptide synthesis. For example, where a dimer with two intramolecular disulfide bonds is desired, the first peptide monomer chain is synthesized with the two cysteine residues of the core sequence protected with a first thiol protecting group [e.g., trityl(Trt), allyloxycarbonyl (Alloc), and 1-(4,4-dimethyl-2,6-dioxocyclohex-1-ylidene)ethyl (Dde) or the like], then the second peptide monomer is synthesized the two cysteine residues of the core sequence protected with a second thiol protecting group different from the first thiol protecting group [e.g., acetamidomethyl (Acm), t-butyl (tBu), or the like]. Thereafter, the first thiol protecting groups are removed effecting bisulfide cyclization of the first peptide monomer, and then the second thiol protecting groups are removed effecting bisulfide cyclization of the second peptide monomer.

Other embodiments of this invention provide for analogues of these disulfide derivatives in which one of the sulfurs has been replaced by a CH₂ group or other isotere for sulfur. These analogues can be prepared from the compounds of the present invention, wherein each peptide monomer contains at least one C or homocysteine residue and an α-amino-γ-butyric acid in place of the second C residue, via an intramolecular or intermolecular displacement, using methods known in the art [See, e.g., Barker, et al. (1992) J. Med. Chem. 35:2040-2048 and Or, et al. (1991) J. Org. Chem. 56:3146-3149]. One of skill in the art will readily appreciate that this displacement can also occur using other homologs of α-amino-γ-butyric acid and homocysteine.

In addition to the foregoing cyclization strategies, other non-disulfide peptide cyclization strategies can be employed. Such alternative cyclization strategies include, for example, amide-cyclization strategies as well as those involving the formation of thio-ether bonds. Thus, the compounds of the present invention can exist in a cyclized form with either an intramolecular amide bond or an intramolecular thio-ether bond. For example, a peptide may be synthesized wherein one cysteine of the core sequence is replaced with lysine and the second cysteine is replaced with glutamic acid. Thereafter a cyclic peptide monomer may be formed through an amide bond between the side chains of these two residues. Alternatively, a peptide may be synthesized wherein one cysteine of the core sequence is replaced with lysine (or serine). A cyclic peptide monomer may then be formed through a thio-ether linkage between the side chains of the lysine (or serine) residue and the second cysteine residue of the core sequence. As such, in addition to disulfide cyclization strategies, amide-cyclization strategies and thio-ether cyclization strategies can both be readily used to cyclize the compounds of the present invention. Alternatively, the amino-terminus of the peptide can be capped with an α-substituted acetic acid, wherein the α-substituent is a leaving group, such as an α-haloacetic acid, for example, α-chloroacetic acid, α-bromoacetic acid, or α-iodoacetic acid.

Other Modifications

Other modifications of the peptides and peptide-based compounds of the invention include the addition of spacer moieties and/or poly(ethylene glycol) (“PEG”) moieties.

The peptides and peptide-based compounds of the invention further comprise a spacer moiety. In one embodiment the spacer may be incorporated into the peptide during peptide synthesis. For example, where a spacer contains a free amino group and a second functional group (e.g., a carboxyl group or an amino group) that enables binding to another molecular moiety, the spacer may be conjugated to the solid support.

In one embodiment, a spacer containing two functional groups is first coupled to the solid support via a first functional group. Next, a lysine linker moiety having two functional groups capable of serving as initiation sites for peptide synthesis and a third functional group (e.g., a carboxyl group or an amino group) that enables binding to another molecular moiety is conjugated to the spacer via the spacer's second functional group and the linker's third functional group. Thereafter, two peptide monomers may be synthesized directly onto the two reactive nitrogen groups of the linker moiety in a variation of the solid phase synthesis technique. For example, a solid support coupled spacer with a free amine group may be reacted with a lysine linker via the linker's free carboxyl group.

In alternate embodiments the spacer may be conjugated to the peptide dimer after peptide synthesis. Such conjugation may be achieved by methods well established in the art. In one embodiment, the linker contains at least one functional group suitable for attachment to the target functional group of the synthesized peptide. For example, a spacer with a free amine group may be reacted with a peptide's C-terminal carboxyl group. In another example, a linker with a free carboxyl group may be reacted with the free amine group of a lysine amide.

In recent years, water-soluble polymers, such as polyethylene glycol (PEG), have been used for the covalent modification of peptides of therapeutic and diagnostic importance. Attachment of such polymers is thought to enhance biological activity, prolong blood circulation time, reduce immunogenicity, increase aqueous solubility, and enhance resistance to protease digestion. For example, covalent attachment of PEG to therapeutic polypeptides such as interleukins [Knauf, et al. (1988) J. Biol. Chem. 263; 15064; Tsutsumi, et al. (1995) J. Controlled Release 33:447), interferons (Kita, et al. (1990) Drug Des. Delivery 6:157), catalase (Abuchowski, et al. (1977) J. Biol. Chem. 252:582), superoxide dismutase (Beauchamp, et al. (1983) Anal. Biochem. 131:25), and adenosine deaminase (Chen, et al. (1981) Biochim. Biophy. Acta 660:293), has been reported to extend their half life in vivo, and/or reduce their immunogenicity and antigenicity.

The peptides and peptide-based compounds of the invention may comprise a polyethylene glycol (PEG) moiety, which is covalently attached to the branched tertiary amide linker or the spacer of the peptide dimer via a carbamate linkage or via an amide linkage. An example of PEG used in the present invention is linear, unbranched PEG having a molecular weight of about 20 kiloDaltons (20K) to about 40K (the term “about” indicating that in preparations of PEG, some molecules will weigh more, some less, than the stated molecular weight). Preferably, the PEG has a molecular weight of about 30K to about 40K.

Another example of PEG used in the present invention is linear PEG having a molecular weight of about 10K to about 60K (the term “about” indicating that in preparations of PEG, some molecules will weigh more, some less, than the stated molecular weight). Preferably, the PEG has a molecular weight of about 20K to about 40K. More preferably, the PEG has a molecular weight of about 20K.

Another example of PEG used in the present invention is a bifunctional PEG or a trifunctional PEG. A bifunctional PEG is covalently attached to two peptide monomer sequences of the invention. A trifunctional PEG is covalently attached to three peptide monomer sequences of the invention.

The illustrative examples described above are not intended to be limiting. One of ordinary skill in the art will appreciate that a variety of methods for covalent attachment of a broad range of PEG is well established in the art. As such, peptides and peptide-based compounds to which PEG has been attached by any of a number of attachment methods known in the art are encompassed by the present invention. Non-limiting examples of such spacer moiety and PEG moiety modifications are such as those described in U.S. Pat. No. 7,084,245, or 4,179,337 and U.S. Publication Nos. US 2005-0107297 A1 to Holmes, et al., US-2007-0032408 A1 to Holmes, et al., US 2005-0107297 A1 to Holmes et al., US 2007-0027074 A1 to Holmes, et al., and US 2007-0032408 A1 to Holmes, et al.

Use of Agonist and Antagonist Peptides and Peptide-Based Compounds of the Invention

The peptides of the invention can also be utilized as commercial reagents for various medical research and diagnostic purposes. Such uses can include but are not limited to: (1) use as a calibration standard for quantitating the activities of candidate TRAIL receptor agonists or antagonists in a variety of functional assays; (2) use in co-crystallization with TRAIL R2, i.e., crystals of the peptides of the present invention bound to the TRAIL R2 may be formed, enabling determination of receptor/peptide structure by X-ray crystallography; (3) use to measure the capacity of TRAIL ligand to protect against cancer or induce characteristic features of inflammatory diseases, such as, but not limited to, asthma, in various disease and disorder models (4), use related to labeling the peptides of the invention with a radioactive chromophore; and (5) other research and diagnostic applications wherein the agonist or antagonist activity of candidate TRAIL R2-binding peptides or peptide compounds are conveniently calibrated against a known quantity of a TRAIL R2 agonist or antagonist, and the like.

In yet another aspect of the present invention, methods of treatment and manufacture of a medicament are provided. The peptides and peptide-based compounds of the invention may be administered to mammals, including humans, to treat cancer (using TRAIL agonists) or asthma (using TRAIL antagonists). Thus, the present invention encompasses methods for therapeutic treatment of disorders that benefit from the modulation of TRAIL receptor signaling, which methods comprise administering a peptide agonist or antagonist of the invention in amounts sufficient to stimulate TRAIL R2 and thus induce the desired effect in vivo. In other embodiments, the peptides and peptide-based compounds of the invention may be used in combinational therapies.

Use of Agonist Peptides and Peptide-Based Compounds of the Invention

The agonist peptides and peptide-based compounds of the invention are useful in vitro as tools for understanding the biological role of TRAIL. For example, peptide agonists of the invention may be used to conduct in vitro cancer research. The effect of TRAIL R2 agonist peptides of the invention on different cancer cell lines, such as, for example Jurkat cells, may be studied or determined. Specifically, TRAIL peptide agonists may be used to study TRAIL R2 signaling pathways in order to identify new targets for the induction of apoptosis in cancer cells. In vitro systems may also used to test the effectiveness of peptides and peptide compound agonists of the invention in combination with other anti-cancer agents, as described below.

The agonist peptides and peptide-based compounds of the invention are useful therapeutic agents in vivo, as they may be used to treat a variety of malignant tumors in individuals with cancer alone or in combination with other therapeutic agents. These agonist peptides are particularly useful for the treatment of malignant tumors that have mutated the p53 gene. Tumors such as these are no longer susceptible to induction of apoptosis via p53, and therefore, are resistant to traditional therapies such as radio- and chemotherapy. It would therefore be beneficial to have agonist peptides, such as those provided by the present invention, that induce apoptosis in tumor cells via a p53-independent mechanism.

In other embodiments, the agonist peptides and peptide-based compounds of the invention are useful to kill or inhibit the growth of cancer cells. The cancer cells may be derived from any cell type including, without limitation, epidermal, epithelial, endothelial or mesodermal cells. The tumor cells may be derived from solid or non-solid tumors including, but not limited to, leukemia, sarcoma, carcinoma, lymphoma, adenocarcinoma, melanoma, multiple myeloma, glioblastoma, choriocarcinoma, Wilms tumor, Kaposi or cervical intraepithelial neoplasia.

In some embodiments of the invention, a TRAIL peptide agonist is used to treat lung cancer, bone cancer, pancreatic cancer, skin cancer, cancer of the head and neck, cutaneous or intraocular melanoma, uterine cancer, ovarian cancer, rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas).

A TRAIL peptide agonist of the current invention that has apoptotic activity is used to treat brain, lung, squamous cell, bladder, gastric, pancreatic, breast, head, neck, liver, renal, ovarian, prostate, colorectal, esophageal, gynecological, nasopharynx, or thyroid cancers, melanomas, lymphomas, leukemias, multiple myelomas, choriocarcinoma, Kaposi or cervical intraepithelial neoplasia

Furthermore, the agonist peptides and peptide-based compounds of the invention may also be co-administered for combinational therapy with other anti-cancer agents including, but not limited to, Adriamycin® (doxorubicin), Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Many other anti-cancer drugs, alkylating agents, and chemotherapeutic agents are possible, and are described, for example, in U.S. Pat. No. 7,385,084 to Koya, et al.

Use of the Antagonist Peptides and Peptide-Based Compounds of the Invention

The antagonist peptides and peptide-based compounds of the invention are useful in vitro as tools for understanding the biological role of TRAIL in inflammatory processes, such as, but not limited to, asthma. As discussed above, TRAIL is thought to mediate exacerbation of asthma, in part, by prolonging survival of eosinophils. Thus, it would be useful to study in vitro the effect of antagonist peptides and peptide-based compounds of the invention on primary eosinophils isolated from asthmatic individuals in the presence of TRAIL ligand. Furthermore, the antagonist peptides of the invention could be used to test the effect of TRAIL ligand on other lymphocytes that are thought to be involved in inflammatory disease pathogenesis, for example, but not limited to, asthma.

In other embodiments, peptide antagonists of the invention may be used as blocking reagents in random peptide screening, i.e., in looking for new families of TRAIL R2 peptide ligands, the peptides can be used to block recovery of TRAIL peptides of the present invention.

The antagonist peptides and peptide-based compounds of the invention are especially useful in vivo for the treatment and/or prevention of asthma. TRAIL R2 peptide antagonists may be administered to a patient suffering from asthma to inhibit the symptoms of asthma, including airway hyperresponsiveness and inflammation, recruitment of lymphocytes to the airways, and recruitment and/or prolonged survival of eosinophils.

Combinational therapies are also envisioned whereby activity of the TRAIL antagonist peptides or peptide-based compounds of the invention are supplemented by the addition of one or more other pharmacologically active compounds that enhance or add to their overall ameliorative or preventative effect. Examples include the use of additional antihistamines such as Claritin® anti-histamine and/or anti-IL 9, -IL-4, -IL-5, or -IL-13 receptor antibodies or antagonists. Anti-inflammatory agents that may be administered with the compounds of the invention include, but are not limited to, corticosteroids (e.g., betamethasone, budesonide, cortisone, dexamethasone, hydrocortisone, methylprednisolone, prednisolone, prednisone, and triamcinolone), nonsteroidal anti-inflammatory drugs (e.g., diclofenac, diflunisal, etodolac, fenoprofen, floctafenine, flurbiprofen, ibuprofen, indomethacin, ketoprofen, meclofenamate, mefenamic acid, meloxicam, nabumetone, naproxen, oxaprozin, phenylbutazone, piroxicam, stilindac, tenoxicam, tiaprofenic acid, and tolmetin.), as well as antihistamines, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap. The above-described agents are well-known agents used for the treatment of asthma and other inflammatory disorders [See, e.g., U.S. Patent Application No. 2006-0014680 to Xu, et al.] Other known asthma medications that may be administered with the peptides and peptide-based compounds of the invention include cromolyn, theophylline, and nedocromil.

In accordance with the present invention there may be employed conventional molecular biology, microbiology, protein expression and purification, antibody, and recombinant DNA techniques within the skill of the art. Such techniques are explained fully in the literature. See, e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.; Nucleic Acid Hybridization, Hames & Higgins eds. (1985); Transcription And Translation, Hames & Higgins, eds. (1984); Animal Cell Culture Freshney, ed. (1986); Immobilized Cells And Enzymes, IRL Press (1986); Perbal, A Practical Guide To Molecular Cloning (1984); and Harlow and Lane. Antibodies: A Laboratory Manual (Cold Spring Harbor Laboratory Press: 1988).

Pharmaceutical Compositions

In yet another aspect of the present invention, pharmaceutical compositions of TRAIL R2 agonist peptides and peptide-based compounds of the invention are provided. In another aspect of the present invention, pharmaceutical compositions of the TRAIL R2 antagonists and peptide-based compounds of the invention are provided. Conditions alleviated or modulated by the administration of such compositions include those indicated above. Such pharmaceutical compositions may be for administration by oral, parenteral (intramuscular, intraperitoneal, intravenous (IV) or subcutaneous injection), transdermal (either passively or using iontophoresis or electroporation), transmucosal (nasal, vaginal, rectal, or sublingual) routes of administration or using bioerodible inserts and can be formulated in dosage forms appropriate for each route of administration.

In certain embodiments, active compounds comprising a peptide or peptide-based compound of the invention may be prepared with a carrier that will protect the peptide or peptide-based compound of the invention against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems (J. R. Robinson, ed., Marcel Dekker, Inc., New York, 1978).

In general, comprehended by the invention are pharmaceutical compositions consisting of an effective amounts of a TRAIL R2 agonist or alternatively, antagonist peptide, or derivative products, of the invention together with pharmaceutically acceptable diluents, preservatives, solubilizers, emulsifiers, adjuvants and/or carriers. Diluents of various buffer content (e.g., Tris-HCl, acetate, phosphate), pH and ionic strength; additives such as detergents and solubilizing agents (e.g., Tween® 20, Tween® 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder (e.g., lyophilized) form.

Oral Delivery

Contemplated for use herein are oral solid dosage forms, which are described generally in Remington's Pharmaceutical Sciences, 18th Ed. 1990 (Mack Publishing Co. Easton Pa. 18042) at Chapter 89, which is herein incorporated by reference. Solid dosage forms include tablets, hard or soft-shell gelatin capsules, pills, troches or lozenges, cachets, pellets, powders, or granules; or the compound or pharmaceutical compound may be incorporated directly into the subject's diet. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (See, e.g., U.S. Pat. No. 5,013,556). A description of possible solid dosage forms for the therapeutic is given by Marshall, K. In: Modern Pharmaceutics Edited by G. S. Banker and C. T. Rhodes Chapter 10, 1979, herein incorporated by reference. In general, the formulation will include the peptides and peptide-based compounds of the invention (or chemically modified forms thereof) and inert ingredients which allow for protection against the stomach environment, and release of the biologically active material in the intestine.

Also contemplated for use herein are liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

The peptides may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. As discussed in the foregoing comments, PEGylation is an example of a chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane [see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al. (1982) J. Appl. Biochem. 4:185-189].

For oral formulations, the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the peptide (or derivative) or by release of the peptide (or derivative) beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (i.e. powder), for liquid forms a soft gelatin shell may be used. The shell material of cachets could be thick starch or other edible paper. For pills, lozenges, molded tablets or tablet triturates, moist massing techniques can be used.

The peptides (or derivatives) and peptide-based compounds of the invention can be included in the formulation as fine multiparticulates in the form of granules or pellets of particle size about 1 mm. The formulation of the material for capsule administration could also be as a powder, lightly compressed plugs, or even as tablets. These therapeutic agents can be prepared by compression.

Colorants and/or flavoring agents may also be included. For example, the peptide (or derivative) may be formulated (such as by liposome or microsphere encapsulation) and then further contained within an edible product, such as a refrigerated beverage containing colorants and flavoring agents.

One may dilute or increase the volume of the peptides (or derivatives) and peptide-based compounds of the invention with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo® diluent, Emdex® dextrate, STA-Rx 1500® starch, Emcompress® binder and Avicell® microcrystalline cellulose.

Disintegrants may be included in the formulation of the therapeutic into a solid dosage form. Materials used as disintegrates include but are not limited to starch, including the commercial disintegrant based on starch, Explotab® disintegrant. Sodium starch glycolate, Amberlite™ resin, sodium carboxymethylcellulose, ultramylopectin, sodium alginate, gelatin, orange peel, acid carboxymethyl cellulose, natural sponge and bentonite may all be used. The disintegrants may also be insoluble cationic exchange resins. Powdered gums may be used as disintegrants and as binders. and can include powdered gums such as agar, Karaya or tragacanth. Alginic acid and its sodium salt are also useful as disintegrants.

Binders may be used to hold the peptide (or derivative) agent or peptide-based compounds of the invention together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the peptide (or derivative).

An antifrictional agent may be included in the formulation of the peptide (or derivative) to prevent sticking during the formulation process. Lubricants may be used as a layer between the peptide (or derivative) and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax™ 4000 and 6000 poly(ethylene glycol).

Glidants that might improve the flow properties of the drug during formulation and to aid rearrangement during compression might be added. The glidants may include starch, talc, pyrogenic silica and hydrated silicoaluminate.

To aid dissolution of the peptide (or derivative) into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethonium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants could be present in the formulation of the protein or derivative either alone or as a mixture in different ratios.

Additives which potentially enhance uptake of the peptide (or derivative) are for instance the fatty acids oleic acid, linoleic acid and linolenic acid.

Controlled release oral formulations may be desirable. The peptide (or derivative) could be incorporated into an inert matrix which permits release by either diffusion or leaching mechanisms, e.g., gums. Slowly degenerating matrices may also be incorporated into the formulation. Some enteric coatings also have a delayed release effect. Another form of a controlled release is by a method based on the Oros® therapeutic system (Alza Corp., Mountain View, Calif.), i.e. the drug is enclosed in a semipermeable membrane which allows water to enter and push drug out through a single small opening due to osmotic effects.

Other coatings may be used for the formulation. These include a variety of sugars which could be applied in a coating pan. The peptide (or derivative) could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan coater or in a fluidized bed or by compression coating.

Parenteral Delivery

Preparations according to this invention for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Preferred routes of parenteral administration are subcutaneously or intramuscularly. A still more preferred route of administration is intravenous administration.

Rectal or Vaginal Delivery

Compositions for rectal or vaginal administration are preferably suppositories which may contain, in addition to the active substance, excipients such as cocoa butter or a suppository wax. Compositions for nasal or sublingual administration are also prepared with standard excipients well known in the art.

Pulmonary Delivery

Also contemplated herein is pulmonary delivery of the TRAIL R2 agonist or antagonist peptides (or derivatives thereof). The agonist or antagonist peptide (or derivative) is delivered to the lungs of a mammal while inhaling and traverses across the lung epithelial lining to the blood stream [see, e.g., Adjei, et al. (1990) Pharmaceutical Research 7:565-569; Adjei, et al. (1990) Int. J. Pharmaceutics 63:135-144 (leuprolide acetate); Braquet, et al. (1989) J. Cardiovascular Pharmacology 13(sup5):143-146 (endothelin-1); Hubbard, et al. (1989) Annals of Internal Medicine, Vol. III, pp. 206-212 (α1-antitrypsin); Smith, et al. (1989) J. Clin. Invest. 84:1145-1146 (α-1-proteinase); Oswein, et al. (1990) “Aerosolization of Proteins”, Proceedings of Symposium on Respiratory Drug Delivery II Keystone, Colo. (recombinant human growth hormone); Debs, et al. (1988) J. Immunol. 140:3482-3488 (interferon-γ and tumor necrosis factor α); and U.S. Pat. No. 5,284,656 to Platz, et al. (granulocyte colony stimulating factor). A method and composition for pulmonary delivery of drugs for systemic effect is described in U.S. Pat. No. 5,451,569 to Wong, et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of this invention are the Ultravent® nebulizer (Mallinckrodt Inc., St. Louis, Mo.); the Acorn II® nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.).

All such devices require the use of formulations suitable for the dispensing of peptide (or derivative). Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified peptides may also be prepared in different formulations depending on the type of chemical modification or the type of device employed.

Formulations suitable for use with a nebulizer, either jet or ultrasonic, will typically comprise peptide (or derivative) dissolved in water at a concentration of about 0.1 to 25 mg of biologically active protein per ml of solution. The formulation may also include a buffer and a simple sugar (e.g., for protein stabilization and regulation of osmotic pressure). The nebulizer formulation may also contain a surfactant, to reduce or prevent surface induced aggregation of the peptide (or derivative) caused by atomization of the solution in forming the aerosol.

Formulations for use with a metered-dose inhaler device will generally comprise a finely divided powder containing the peptide (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing peptide (or derivative) and may also include a bulking agent, such as lactose, sorbitol, sucrose, or mannitol in amounts which facilitate dispersal of the powder from the device, e.g., 50 to 90% by weight of the formulation. The peptide (or derivative) should most advantageously be prepared in particulate form with an average particle size of less than 10 mm (or microns), most preferably 0.5 to 5 mm, for most effective delivery to the distal lung.

Nasal Delivery

Nasal delivery of the TRAIL R2 agonist or antagonist peptides (or derivatives) is also contemplated. Nasal delivery allows the passage of the peptide(s) to the blood stream directly after administering the therapeutic product to the nose, without the necessity for deposition of the product in the lung. Formulations for nasal delivery include, but are not limited to, those with dextran or cyclodextran.

Other penetration-enhancers used to facilitate nasal delivery are also contemplated for use with the peptides of the present invention (such as described in International Patent Publication No. WO 2004056314, filed Dec. 17, 2003, incorporated herein by reference in its entirety).

Dosages

For all of the peptides and peptide-based compounds of the invention, as further studies are conducted, information will emerge regarding appropriate dosage levels for treatment of various conditions in various patients, and the ordinary skilled worker, considering the therapeutic context, age, and general health of the recipient, will be able to ascertain proper dosing. The term patient includes human and veterinary subjects. The selected dosage depends upon the desired therapeutic effect, on the route of administration, and on the duration of the treatment desired. Generally dosage levels of 0.001 to 10 mg/kg of body weight daily are administered to mammals. The dosing schedule may vary, depending on the circulation half-life and the formulation used. Dosage regimens can be determined, adjusted, or titrated to provide the optimum desired response (e.g., a therapeutic or prophylactic response) using routine methods. For example, a single bolus can be administered, several divided doses can be administered over time or the dose can be proportionally reduced or increased as indicated by the exigencies of the therapeutic situation.

The therapeutic dose range in the methods of the invention can be 100 milligrams (mg) of agonist compound or 100 milligrams (mg) antagonist compound per 1 kilogram (kg) of body weight of the individual (mg/kg). More particularly, the dose range of 10 mg/kg would be preferred for agonist compounds of the invention and the dose rang of 10 mg/kg would be preferred for antagonist compounds of the invention. Furthermore, a physician may initially use escalating dosages, starting at 1 mg/kg for agonist compounds of the invention, or 1 mg/kg for antagonist compounds of the invention, and then titrate the dosage at approximately 25%-50% increments for each individual being treated.

In certain embodiments, the compositions of the invention may include a “therapeutically effective amount” or a “prophylactically effective amount” of an agonist peptide or peptide-based compound of the invention. In other embodiments, the compositions of the invention may include a “therapeutically effective amount” or a “prophylactically effective amount” of an antagonist peptide or peptide-based compound of the invention. A “therapeutically effective amount” refers to an amount effective, at dosages and for periods of time necessary, to achieve the desired therapeutic result. A therapeutically effective amount of a peptide or peptide-based compound of the invention may vary according to factors such as the disease state, age, sex, and weight of the individual, and the ability of the peptide or peptide-based compound of the invention to elicit a desired response in the individual. A therapeutically effective amount is also one in which any toxic or detrimental effects of the peptide or peptide-based compound are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Another aspect of the present invention provides kits comprising a peptide or peptide-based compound of the invention or a composition comprising such a peptide or peptide-based compound. A kit may include, in addition to the peptide or peptide-based compound, diagnostic or therapeutic agents. A kit can also include instructions for use in a diagnostic or therapeutic method. In a preferred embodiment, the kit includes the peptide or peptide-based compound or a composition comprising it and a diagnostic agent that can be used in a method described below. In another preferred embodiment, the kit includes the peptide or peptide-based compound or a composition comprising it and one or more therapeutic agents that can be used in a method described below.

Additional active compounds also can be incorporated into the compositions. In certain embodiments, a peptide or peptide-based compound of the invention is co-formulated with and/or co-administered with one or more additional therapeutic agents. Such combination therapies may require lower dosages of the peptide or peptide-based compound as well as the co-administered agents, thus avoiding possible toxicities or complications associated with the various monotherapies.

### EXAMPLES

The present invention is also described by means of the following examples. However, the use of examples anywhere in the specification is illustrative of and in no way limits the scope and meaning of the invention or of any exemplified terms. Likewise, the invention is not limited to any particular embodiment described herein. Indeed, many modifications and variations to those skilled in the art upon reading this specification and can be made without departing from its spirit and scope. The invention is therefore to be limited only by the terms of the appended claims along with the full scope of equivalents to which the claims are entitled. The disclosures of all citations, including issued patents, published applications, and scientific articles, in the specification are expressly incorporated herein by reference in their entirety.

It is to be understood that numerical values of binding activities and other parameters reported in the examples, and throughout the entire specification, are approximate. Individual measurements of these parameters may vary, e.g., due to normal experimental error and/or depending on the specific conditions used.

### Example 1

### Discovery and Optimization of TRAIL R2Agonist Peptides

1.1 Discovery of a Novel Peptide Sequence that Binds to TRAIL R2 (Competition Binding Assay)
Peptide agonists for the TRAIL R2 receptor are first identified in binding competition assays using the AlphaQuest® plate reader and AlphaScreen™ assay kit [Perkin-Elmer®, Waltham, Mass.].
Briefly, a biotinylated TRAIL ligand is conjugated to streptavidin-coated “donor” beads, and a TRAIL R2-Fc fusion protein is conjugated to protein A-conjugated “acceptor” beads. Laser excitation (680 nm) of a photosensitizer in the donor bead generates singlet state oxygen molecules, which react with a chemiluminescer in the acceptor bead to generate a detectable light signal (520-620 nm). Because the singlet O₂ molecules have an extremely short half-life (i.e., t ½), a signal is generated only when the donor and acceptor beads are brought into close proximity, by TRAIL binding to the R2 receptor. The presence of unlabeled test peptide that competes with TRAIL for binding to the R2 receptor will prevent this binding, resulting in a measurable decrease in light emission.

In more detail, a serial dilution of peptide agonist or TRAIL ligand (Axxora, San Diego, Calif.) in AlphaQuest® buffer (40 mM HEPES, pH 7.4, 0.1% bovine serum albumin, 0.05% Tween 20, and 1 mM MgCl₂) is prepared in a polypropylene dilution plate, and 4 μL of each is transferred into a white Greiner 384-well assay plate (E&K Scientific, Santa Clara, Calif.) in triplicate. Recombinant human TRAIL R2-Fc fusion protein (R&D Systems, Minneapolis, Minn.) is diluted in AlphaQuest® buffer to a concentration of 600 pM, pre-mixed with 1.9 μL Protein A-coated acceptor beads and 1.9 μL streptavidin-coated donor beads (Perkin-Elmer®, Waltham, Mass.), then 2 μL is added to each well of the assay plate. Biotinylated TRAIL ligand is diluted in AlphaQuest® buffer to a concentration of 20 nM, and 2 μL is added to each well of the assay plate. The plates are sealed with adhesive sealing film and covered with aluminum foil, centrifuged at 600 rpm for 30 seconds and incubated overnight in the dark. To determine the amount of TRAIL ligand binding, the plates are quantified by detecting an emitted light signal at 520-620 nm on an AlphaQuest® instrument (Perkin Elmer, Shelton, Conn.). The raw data are analyzed using GraphPad Prism® software (La Jolla, Calif.) to calculate IC₅₀ values from a 4-parameter logistic equation. Peptide binding specificity for human TRAIL R2 is confirmed by repeating the assay with the following recombinant negative control fusion proteins: human TRAIL R1-Fc, human TRAIL R4-Fc, human RANK-Fc, and mouse TRAIL R2-Fc (R&D Systems, Minneapolis, Minn.).

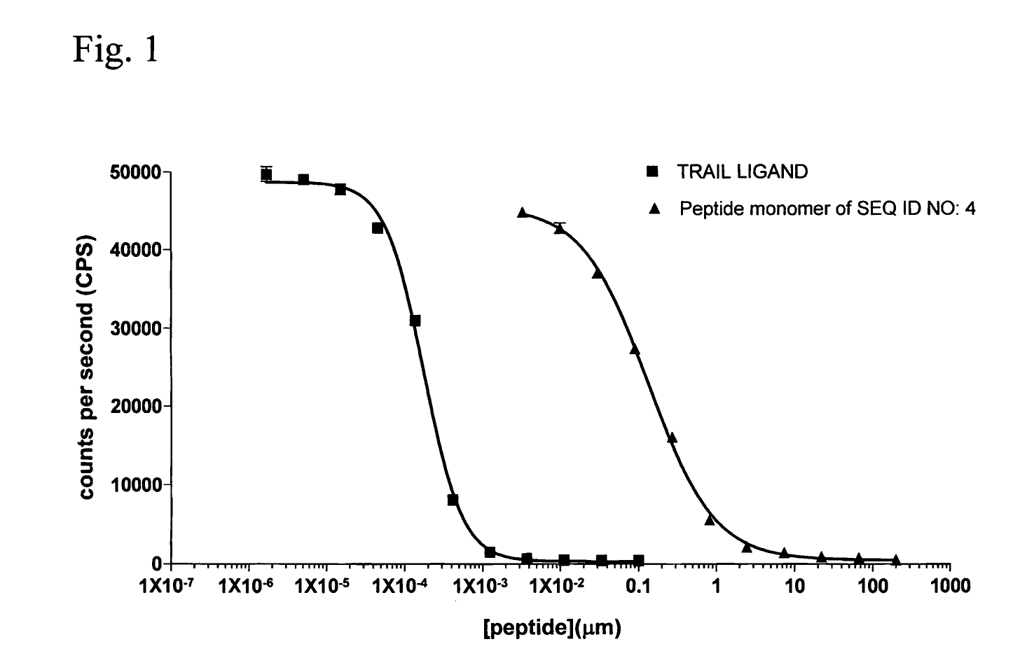

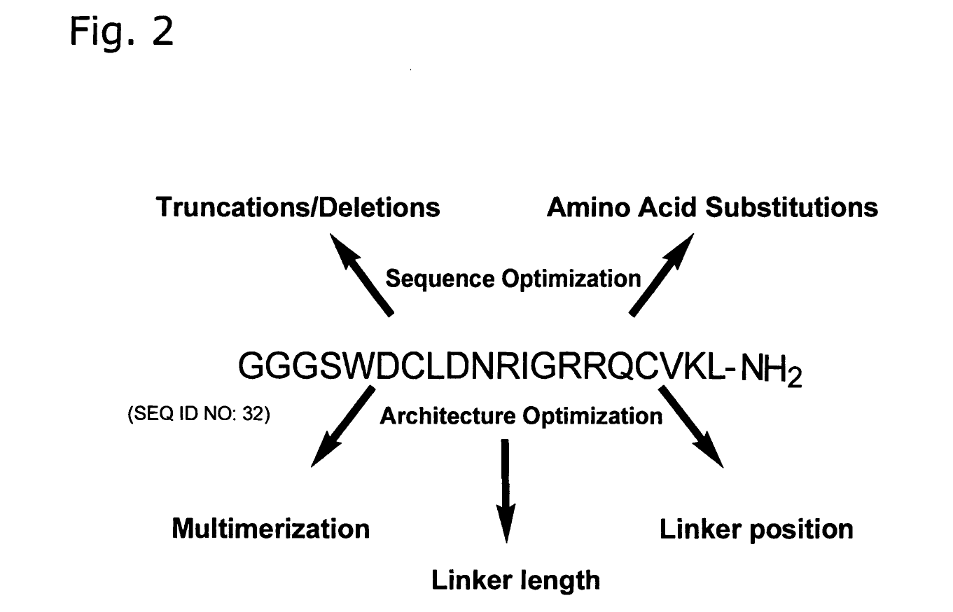

FIG. 1 shows exemplary results of an AlphaQuest® TRAIL R2 receptor binding assay using a test peptide having the amino acid sequence GGGSWDC1DNRIGRRQCVKL (SEQ ID NO: 4). See FIG. 1 for an exemplary binding curve showing that the peptide monomer based on the amino acid sequence of SEQ ID NO: 4 has a binding affinity of about 167 nM compared to the binding affinity of TRAIL ligand, which is about 176 nm. Binding activities of peptide monomers based on the amino acid sequence of SEQ ID NO: 4 from these binding assays have a range of about 67 to about 306 nM. Various modifications and variants of this peptide were generated according to a variety of “hit-to-lead” optimization strategies, illustrated generally in FIG. 2, including sequence optimization (e.g., amino acid truncations, deletions, and/or substitutions), and other modifications such as multimerization, and modifying the length and/or position of various linker moieties joining two or more peptide monomers. FIG. 2 shows a carboxamide at the C-terminal end of the peptide monomer sequence of the invention, but a skilled worker would recognize and appreciate that the C-terminal end of the peptide monomer sequence of the invention may have, alternatively, a free carboxylic acid.

1.2 HCT-116 Proliferation Assay

The apoptotic activity of test peptides is measured using an HCT-116 proliferation assay. The HCT-116 assay is used to measure the activity of TRAIL R2 agonist peptides using the colon carcinoma cell line HCT-116 (ATCC, Manassas, Va.). HCT-116 cells express the TRAIL R2 receptor, and in the presence of TRAIL ligand, undergo apoptosis. HCT-116 cells are treated with test peptide or TRAIL ligand, the activity of an agonist peptide is measured by comparing the amount of cell apoptosis induced by the test peptide to the amount of apoptosis induced by TRAIL ligand.

In more detail, a peptide homotrimer based on the peptide monomer sequence of SEQ ID NO: 19 (See, FIG. 8B) is tested for functional activity using the HCT-116 proliferation assay. HCT-116 cells are incubated for one to two days in the presence of serially diluted peptide dimers or peptide trimers. HCT-116 cells are obtained from the American Type Culture Collection (ATCC, Manassas, Va.) and are maintained in Growth Medium containing McCoy's 5a medium supplemented with 10% FBS, 100 U/mL penicillin, and 100 U/mL streptomycin (Invitrogen, Carlsbad, Calif.). The cells are resuspended at a density of 3×10⁵ cells/mL in Growth Medium, and 100 μl cells is added to each well of the 96-well flat bottom tissue culture assay plates. The plates are incubated overnight in a 37° C., 5% CO₂ incubator. Serial dilutions of synthetic peptides or TRAIL ligand (Axxora, San Diego, Calif.) are prepared using Growth Medium in a 96-well Deep-Well round bottom tissue culture plate. Eleven (11) μL of the serially diluted peptides or TRAIL ligand is added to the HCT-116 cells in triplicate.

Cell viability is measured using an MTT colorimetric viability assay, a colorimetric assay that analyzes the number of viable cells by measuring the ability of mitochondrial dehydrogenase enzyme in the viable cells to cleave tetrazolium salts added to the culture medium [See, Mosmann (1983) J. Immunol. Methods 65:55-63; Muhlenbeck et al., (2000) Journal of Biological Chemistry 275: 32208-32213]. Using this assay, cell proliferation is assessed after 24-48 hours by adding 10 μL of MTT stock solution (ATCC, Manassas, Va.) to the wells. The plates are incubated in a 37° C., 5% CO₂ incubator for another 2-4 hours, followed by the addition of 100 μL of 20% SDS with 0.01 N HCl. After incubation overnight in a 37° C., 5% CO₂ incubator, the plates are quantified by reading the optical density at 595 nm (OD₅₉₅) in a SpectraMax M5 microplate reader (Molecular Devices, Sunnyvale, Calif.). The data are analyzed using GraphPad® Prism software (La Jolla, Calif.) to calculate IC₅₀ values from a 4-parameter logistic equation.

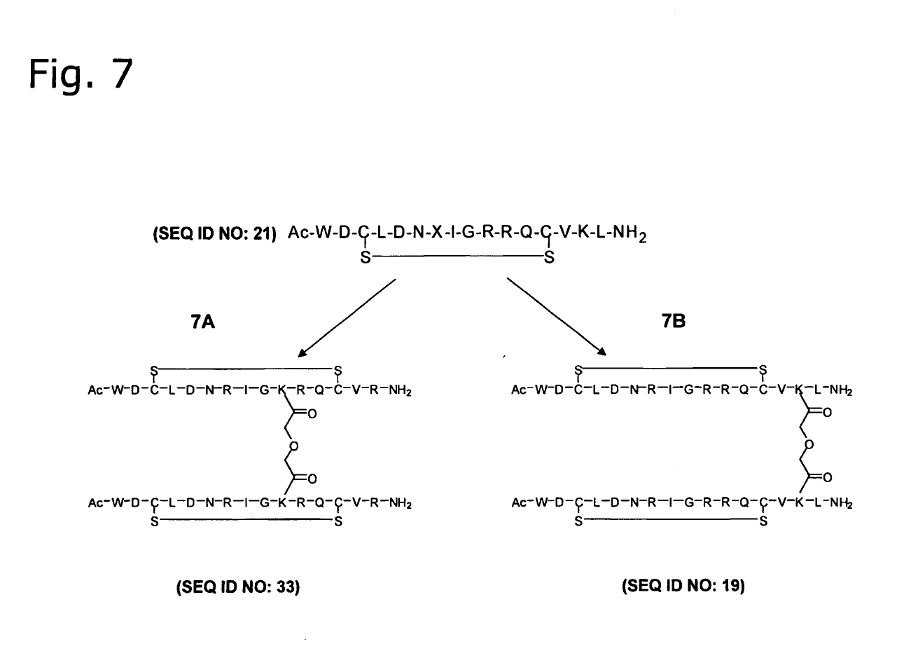

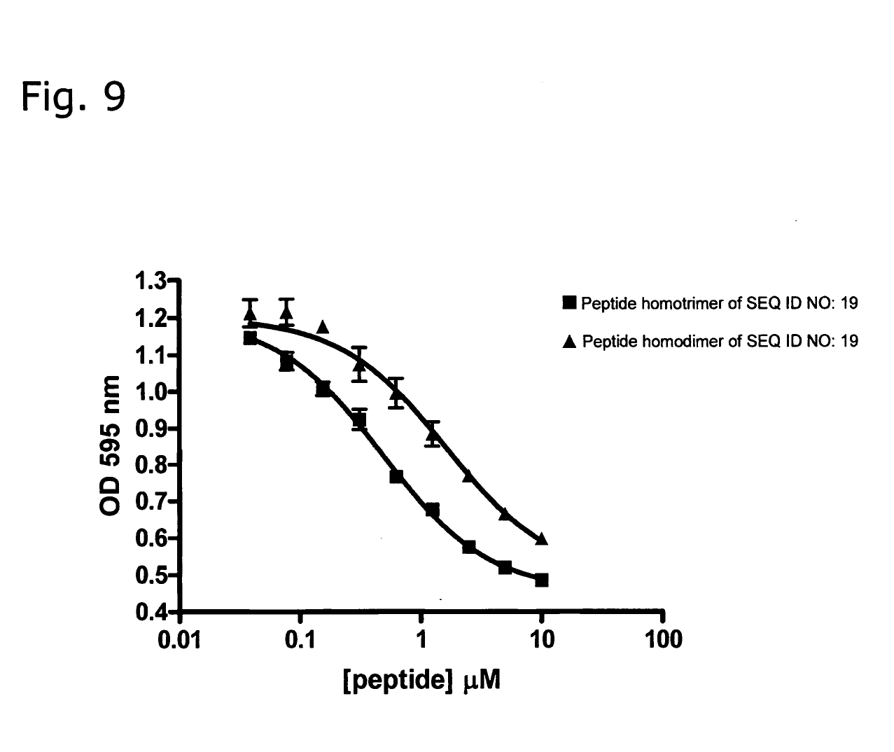

Apoptotic activity of the peptide homotrimeric construct illustrated in FIG. 8B is evaluated in an HCT-116 proliferation assay as described in Section 1.2 above. A homodimer construct based on the same amino acid sequence, which is illustrated in FIG. 7A, is also prepared and evaluated in the same assay. The results of those assays are plotted in FIG. 9. The dimeric construct is determined to have an EC₅₀ value of about 1620 nM. The trimer construct is determined to have substantially greater apoptotic activity, with an EC₅₀ value of about 480 nM. Hence, the trimerization of peptide monomer agonists of the invention can greatly enhance apoptotic activity.

1.3 Jurkat Proliferation Assay

The apoptotic activity of test peptides is measured using a Jurkat proliferation assay. Jurkat cells have been identified as a TRAIL-sensitive cell line [Pitti et al., (1996), Journal of Biological Chemistry 271: 12687-12690], and therefore undergo apoptosis upon treatment with TRAIL ligand.

In more detail, the apoptotic activity of test peptides of the invention is measured by evaluating the ability of synthetic peptides to induce apoptosis in Jurkat cells, compared to TRAIL ligand, as measured by the MTT assay described above (See, Section 1.2). Jurkat cells are obtained from the American Type Culture Collection (ATCC, Manassas, Va.) and are maintained in RPMI-1640 medium supplemented with 10% FBS, 100 U/mL penicillin, and 100 U/mL streptomycin (Growth Medium). Serial dilutions of synthetic peptides or TRAIL ligand (Axxora, San Diego, Calif.) are prepared using Growth Medium in a 96-well Deep-Well round bottom tissue culture plate. Fifty (50) μL of the serially diluted peptides or TRAIL ligand is added to a 96-well flat bottom tissue culture assay plate in triplicate. Cells are resuspended at a density of 1×10⁶ cells/mL in Growth Medium, and 50 μl cells is added to each well of the assay plates. The plates are incubated in a 37° C., 5% CO₂ incubator for 24-48 hours. Proliferation is assessed by adding 10 μL of MTT stock solution (ATCC, Manassas, Va.) to the wells. The plates are incubated in a 37° C., 5% CO₂ incubator for another 2-4 hours, followed by the addition of 100 μL of 20% SDS with 0.01 N HCl. After incubation overnight in a 37° C., 5% CO₂ incubator, the plates are quantified by reading the optical density at a wavelength of 595 nm (OD₅₉₅) in a SpectraMax® M5 microplate reader (Molecular Devices, Sunnyvale, Calif.). The data are analyzed using GraphPad Prism® software (La Jolla, Calif.) to calculate IC₅₀ values from a 4-parameter logistic equation.

To further evaluate effects of peptide dimerization on TRAIL R2 agonists' activity, homodimeric constructs of the peptide sequence set forth in SEQ ID NO: 19 are prepared with DIG, IPA and DOD linker moieties, respectively, and their activity is evaluated in a Jurkat proliferation assay, described above in this section. Constructs of peptide homodimers based on the peptide monomer amino acid sequences of SEQ ID NOs: 29-31 are also prepared with a DIG linker moiety, and their activities are evaluated in the same assay.

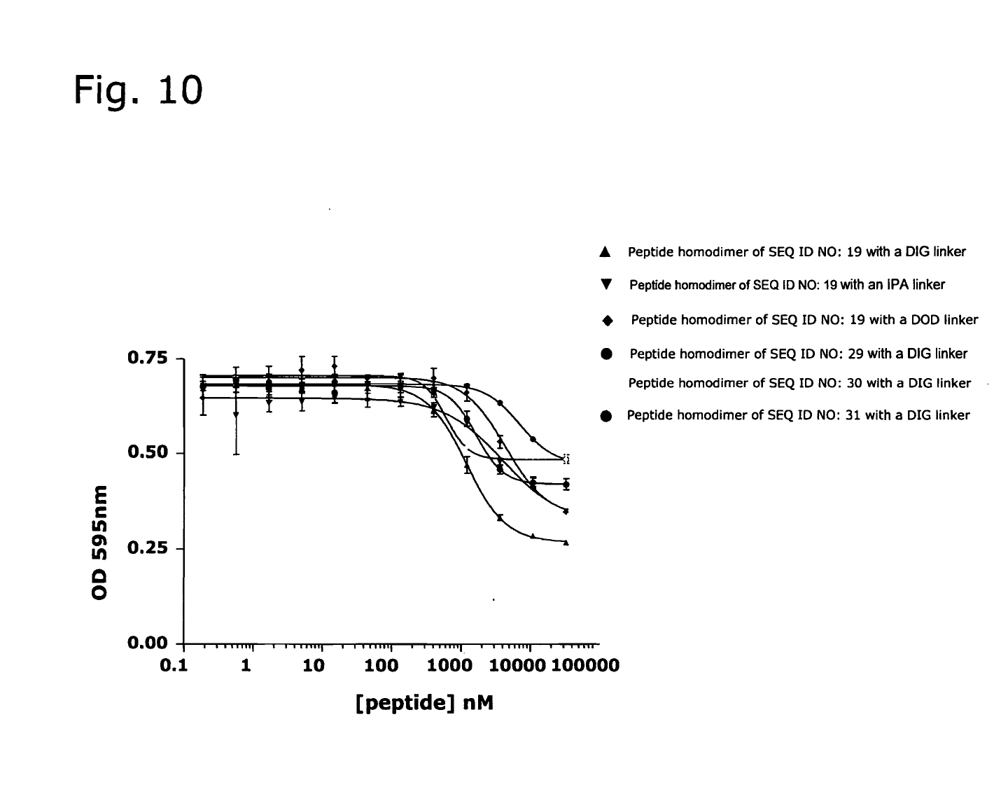

As shown in FIG. 10, the ability of peptide homodimer constructs, each based on one of the peptide monomer amino acid sequences of SEQ ID NOs: 19 or 29-31, to induce apoptosis in Jurkat cells using the Jurkat proliferation assay is calculated as the EC₅₀. In Jurkat cell proliferation assays, the peptide homodimer constructs of the invention based on the peptide monomer amino acid sequence of SEQ ID NO: 19 have an EC₅₀ value having a range of about 0.945 μM to about 7.0 μM (with a DIG linker); a range of about 1400 nM to about 4024 nM (with an IPA linker); and a range of about 2000 nM to about 4413 nM (with a DOD linker); whereas the dimer construct based on the peptide monomer amino acid sequence of SEQ ID NO: 29 has an EC₅₀ value having a range of about 1300 nM to about 1663 nM; the peptide dimer construct based on the peptide monomer amino acid sequence of SEQ ID NO: 30 has an EC₅₀ value having a range of about 611 nM to about 1088 nM; and the peptide dimer construct based on the peptide monomer amino acid sequence of SEQ ID NO: 31 has an EC₅₀ value having a range of about >22,000 nM to about >33,000 nM. Hence, the dimer constructs shown in FIG. 10 are effective agonists of TRAIL R2.

1.4 TRAIL R2 Binding Assay-Truncation Analysis

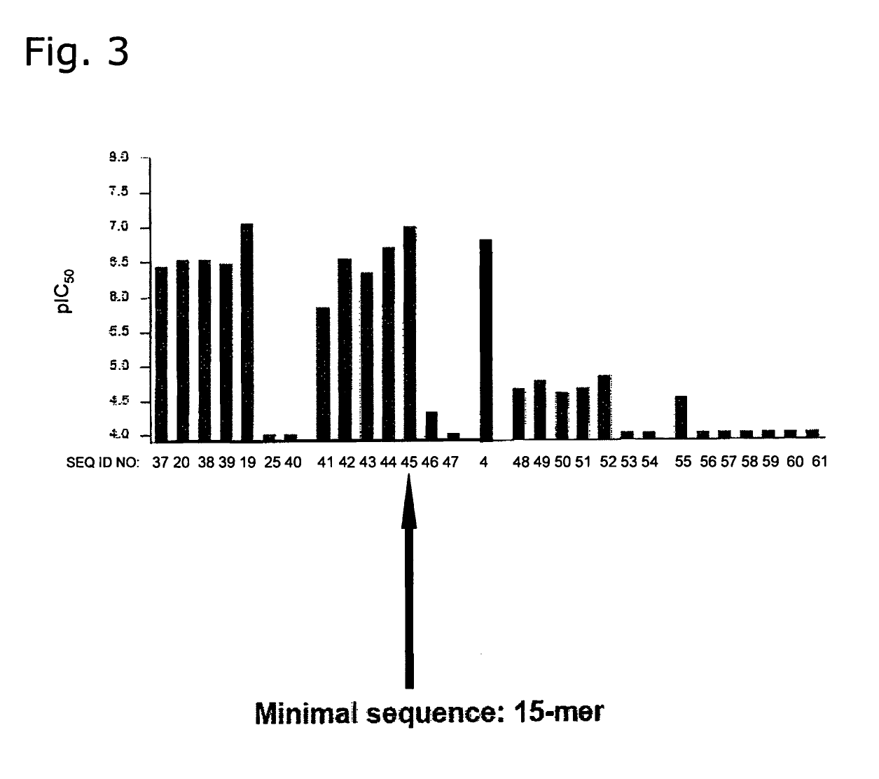

Truncated peptides based on the amino acid sequence set forth in SEQ ID NO: 4 are generated using standard peptide synthesis techniques. Table 5 below shows the amino acid sequences of the truncated peptides that are tested in a competition binding assay. The truncated peptides are serially diluted and are tested for their ability to bind to TRAIL R2 using the AlphaQuest® competition binding assay, described above. The binding activity of the truncated peptides, as set forth in Table 5 below, are compared to the peptide monomer based on the amino acid sequence of SEQ ID NO: 4 in its entirety (i.e. it is not truncated). FIG. 3 shows a graph plotting the measured binding affinities (pIC₅₀) of the truncated peptides, which are sequentially truncated one amino acid at a time from the N-terminus. Truncation of the amino acid sequence generally enhances binding affinity compared to the original peptide sequence of 19 amino acids. However, a minimum sequence of about 15 amino acids is necessary to maintain a binding activity having an IC₅₀ in the range of about 100 nM to about 1000 nM (for pIC₅₀: the range is about 6 nM to about 7 nM).

TABLE 5

| SEQ ID | | | | | | | | | | | | | | | | | | | | | | |
|---|---|---|---|---|---|---|---|---|---|---|---|---|---|---|---|---|---|---|---|---|---|---|
| NO | SEQUENCES BASED ON THE AMINO ACID SEQUENCE OF SEQ ID NO: 4 | | | | | | | | | | | | | | | | | | | | | |
| 37 | Ac | G | G | G | S | W | D | C* | L | D | N | R | I | G | R | R | Q | C* | V | K | L | NH2 |
| 20 | Ac | G | G | S | W | D | C* | L | D | N | R | I | G | R | R | Q | C* | V | K | L | NH2 | |
| 38 | Ac | G | S | W | D | C* | L | D | N | R | I | G | R | R | Q | C* | V | K | L | NH2 | | |
| 39 | Ac | S | W | D | C* | L | D | N | R | I | G | R | R | Q | C* | V | K | L | NH2 | | | |
| 19 | Ac | W | D | C* | L | D | N | R | I | G | R | R | Q | C* | V | K | L | NH2 | | | | |
| 25 | Ac | D | C* | L | D | N | R | I | G | R | R | Q | C* | V | K | L | NH2 | | | | | |
| 40 | Ac | C* | L | D | N | R | I | G | R | R | Q | C* | V | K | L | NH2 | | | | | | |
| 41 | Ac | G | G | G | S | W | D | C* | L | D | N | R | I | G | R | R | Q | C* | V | K | NH2 | |
| 42 | Ac | G | G | S | W | D | C* | L | D | N | R | I | G | R | R | Q | C* | V | K | NH2 | | |
| 43 | Ac | G | S | W | D | C* | L | D | N | R | I | G | R | R | Q | C* | V | K | NH2 | | | |
| 44 | Ac | S | W | D | C* | L | D | N | R | I | G | R | R | Q | C* | V | K | NH2 | | | | |
| 45 | Ac | W | D | C* | L | D | N | R | I | G | R | R | Q | C* | V | K | NH2 | | | | | |
| 46 | Ac | D | C* | L | D | N | R | I | G | R | R | Q | C* | V | K | NH2 | | | | | | |
| 47 | Ac | C* | L | D | N | R | I | G | R | R | Q | C* | V | K | NH2 | | | | | | | |
| 48 | Ac | G | G | G | S | W | D | C* | L | D | N | R | I | G | R | R | Q | C* | V | NH2 | | |
| 49 | Ac | G | G | S | W | D | C* | L | D | N | R | I | G | R | R | Q | C* | V | NH2 | | | |
| 50 | Ac | G | S | W | D | C* | L | D | N | R | I | G | R | R | Q | C* | V | NH2 | | | | |
| 51 | Ac | S | W | D | C* | L | D | N | R | I | G | R | R | Q | C* | V | NH2 | | | | | |
| 52 | Ac | W | D | C* | L | D | N | R | I | G | R | R | Q | C* | V | NH2 | | | | | | |
| 53 | Ac | D | C* | L | D | N | R | I | G | R | R | Q | C* | V | NH2 | | | | | | | |
| 54 | Ac | C* | L | D | N | R | I | G | R | R | Q | C* | V | NH2 | | | | | | | | |
| 55 | Ac | G | G | G | S | W | D | C* | L | D | N | R | I | G | R | R | Q | C* | NH2 | | | |
| 56 | Ac | G | G | S | W | D | C* | L | D | N | R | I | G | R | R | Q | C* | NH2 | | | | |
| 57 | Ac | G | S | W | D | C* | L | D | N | R | I | G | R | R | Q | C* | NH2 | | | | | |
| 58 | Ac | S | W | D | C* | L | D | N | R | I | G | R | R | Q | C* | NH2 | | | | | | |
| 59 | Ac | W | D | C* | L | D | N | R | I | G | R | R | Q | C* | NH2 | | | | | | | |
| 60 | Ac | D | C* | L | D | N | R | I | G | R | R | Q | C* | NH2 | | | | | | | | |
| 61 | Ac | C* | L | D | N | R | I | G | R | R | Q | C* | NH2 | | | | | | | | | |

*= Cysteine residue of disulfide bond

1.5 TRAIL R2 Binding Assay-Alanine Scanning

Alanine scanning mutagenesis is performed to identify amino acid residues that are critical for binding to TRAIL R2 by systematically substituting alanine for selected amino acid residues in the original peptide amino acid sequence. The variant peptides thus obtained are then tested for their ability to inhibit TRAIL ligand binding to the R2 receptor, in an AlphaQuest® competition binding assay, as described above.

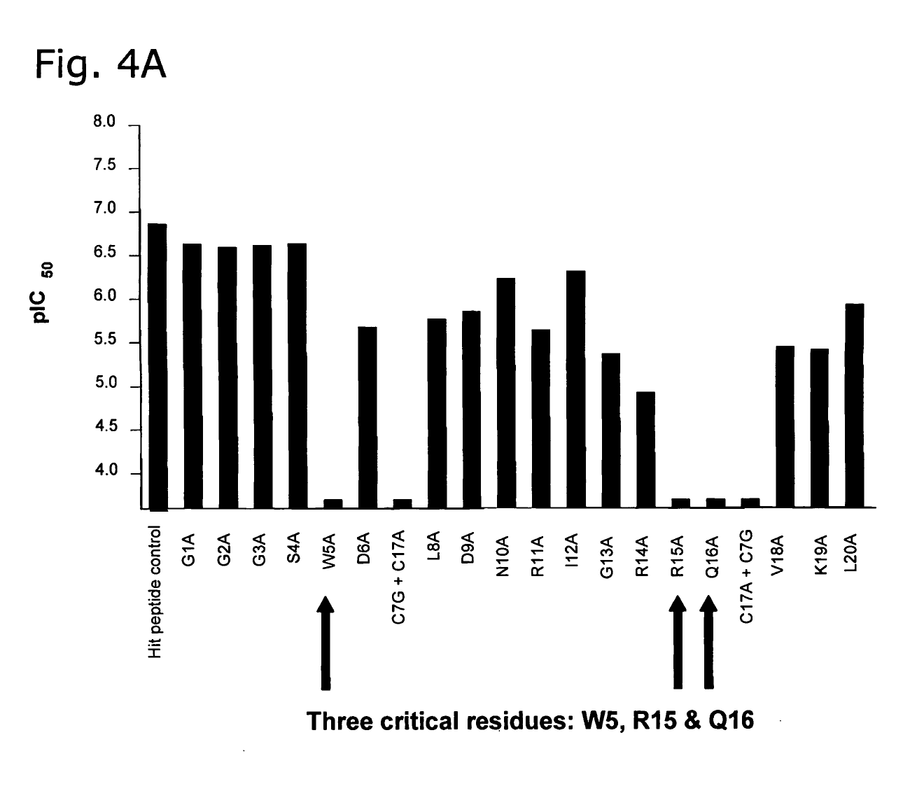

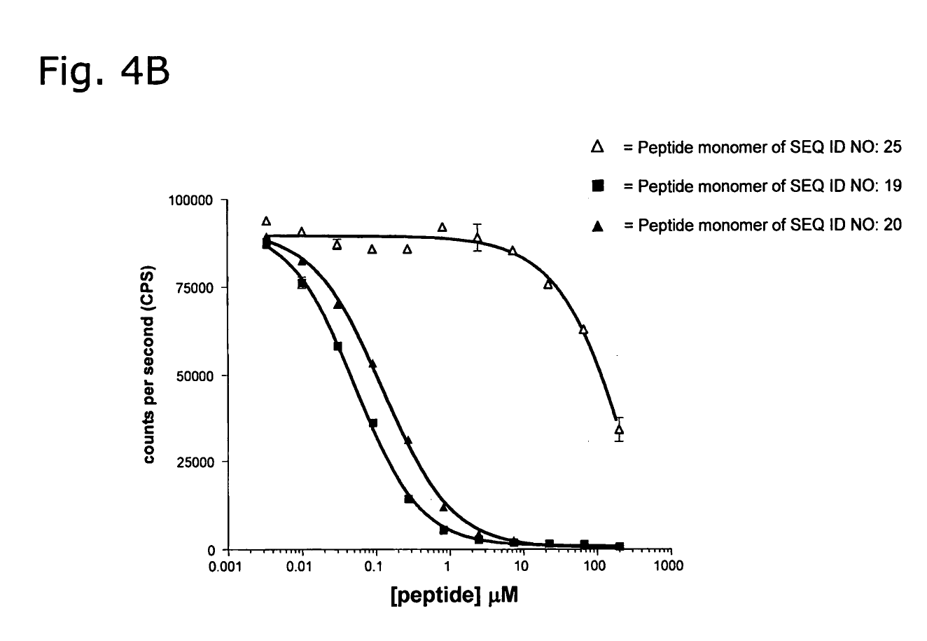

FIG. 4A shows an exemplary graph plotting the negative log IC₅₀ (−log₁₀ IC₅₀) binding affinity (pIC₅₀, which equals −log₁₀ IC₅₀) values for various alanine-substituted derivatives of SEQ ID NO: 4. Hence, for example, the derivative “G1A” indicated on the horizontal axis of FIG. 4A denotes a derivative in which an alanine is substituted for glycine at the first amino acid position in SEQ ID NO: 4. These amino acids were identified—Trp₅, Arg₁₅ and Gln₁₆—whose replacement of alanine results in a significant decrease in binding affinity, which is evidenced by a substantial reduction in pIC₅₀.

FIG. 4B shows an exemplary plot of raw data for another TRAIL R2 binding competition assay with three additional test peptide monomers that are derived from the original peptide amino acid sequence of SEQ ID NO: 4. In the assays for which exemplary data is shown in FIG. 4B, three truncated peptide monomers are tested for their binding affinity to TRAIL R2; a first peptide monomer comprises the amino acid sequence: AcGGSWDCLDNRIGRRQCVKL-NH2 (SEQ ID NO: 20); a second peptide monomer comprises the amino acid sequence: AcWDCLDNRIGRRQCVKL-NH2 (SEQ ID NO: 19); and a third peptide monomer comprises the amino acid sequence: AcDCLDNRIGRRQCVKL-NH2 (SEQ ID NO: 25). An exemplary plot of the analysis of the raw data of binding affinity for the TRAIL R2 receptor indicates in FIG. 4B that the peptide monomer based on the amino acid sequence of SEQ ID NO: 20 has a binding activity (IC₅₀) of about 303 nM, the peptide monomer based on the amino acid sequence of SEQ ID NO: 19 has a binding activity (IC₅₀) of about 49 nM and a range of about 15 nM to about 104 nM, and the peptide monomer based on the amino acid sequence of SEQ ID NO: 25 has a binding activity (IC₅₀) of about or greater than 100 μM. Hence, deleting the N-terminal residue of SEQ ID NO: 4 can give rise to variant peptides with enhanced TRAIL R2 binding affinity.

1.6 Sequence Optimization of Agonist Peptides

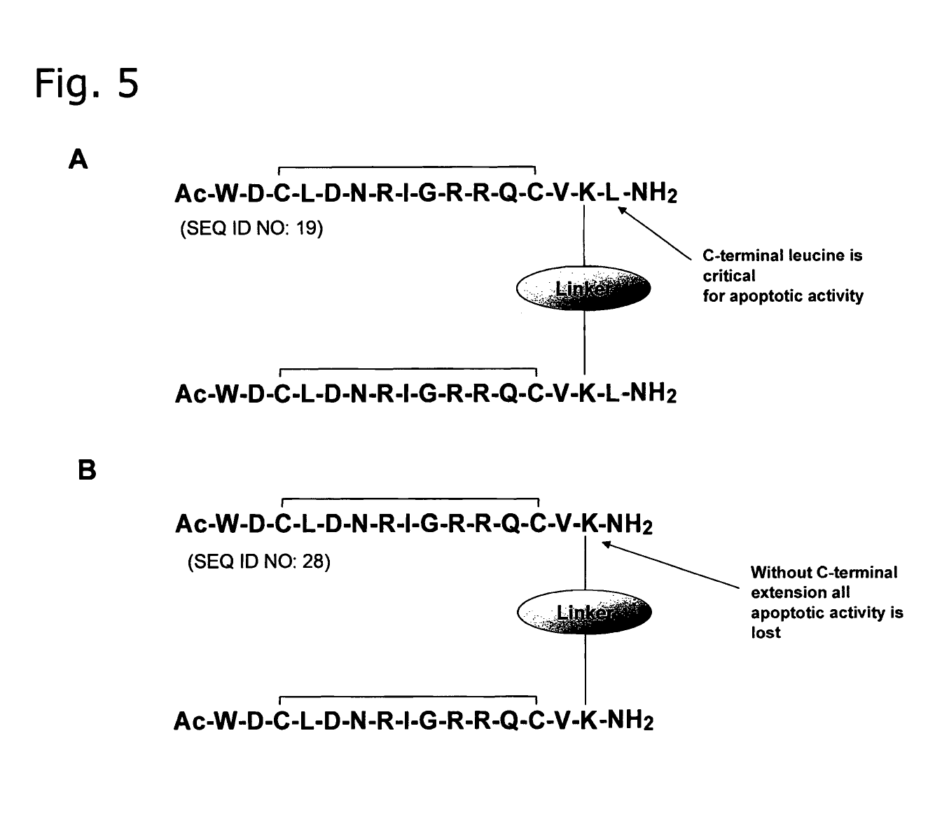

In order to determine whether the C-terminal leucine is important for the binding and functional activity of the peptide monomer based on the amino acid sequence of SEQ ID NO: 19 (AcWDCLDNRIGRRQCVKL-NH2), the leucine residue in position 16 of the peptide monomer based on the amino acid sequence of SEQ ID NO: 19 is deleted from the C-terminus, creating a peptide monomer having the amino acid sequence AcWDCLDNRIGRRQCVK-NH2 (SEQ ID NO: 28) (See, FIG. 5). Binding activity of both peptides to TRAIL R2 is evaluated using the AlphaQuest® competitive binding assay described in Example 1.1, above. An exemplary plot of data from these binding assays indicates that the TRAIL R2 binding activity of the peptide monomer based on the amino acid sequence of SEQ ID NO: 19 has an IC₅₀ value having a range of about 1 nM to about 56 nM. An exemplary plot of data from the analysis of the TRAIL R2 binding assay shows that the IC₅₀ value for the peptide monomer based on the amino acid sequence of SEQ ID NO: 28 is about 80 nM. While the peptide monomer based on the amino acid sequence of SEQ ID NO: 19 exhibits apoptotic activity, no apoptotic activity is observed for the peptide monomer based on the amino acid sequence of SEQ ID NO: 28. Hence, the C-terminal leucine of the peptide monomer based on the amino acid sequence of SEQ ID NO: 19 is critical for apoptotic activity.

1.7 Multimerization of TRAIL R2 Peptides of the Invention Enhance Binding Affinity

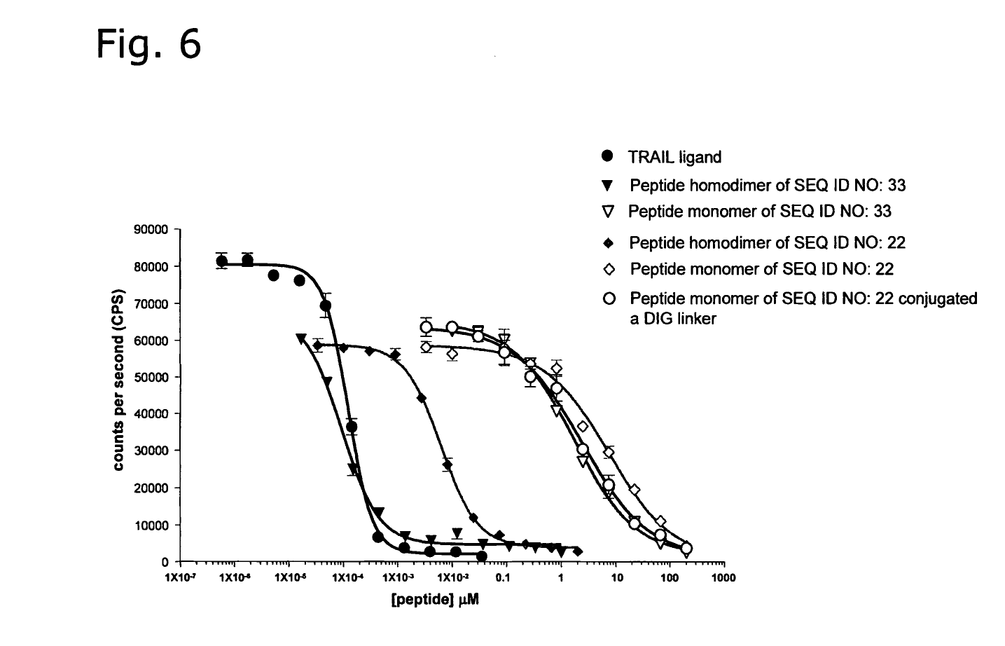

To evaluate effects of multimerization on the binding affinities of TRAIL R2 agonists and antagonists, peptide dimers and other peptide multimers are constructed from TRAIL R2 agonist and antagonist peptide monomer sequences of the invention, and their binding affinities to TRAIL R2 are evaluated in an AlphaQuest® binding competition assay as described above, in Section 1.1. FIG. 6 illustrates results from these binding assays for TRAIL ligand, for peptide monomers based on the amino acid sequences of SEQ ID NO: 33 (AcWDCLDNRIGKRQCVR-NH2) or based on the amino acid sequence of SEQ ID NO: 22 (AcWDCLDRPGRRQCVK-NH2), and for peptide homodimers based on monomer amino acid sequences of SEQ ID NO: 33 or SEQ ID NO: 22, both of which are conjugated with a DIG linker moiety. A peptide monomer based on the amino acid sequence of SEQ ID NO: 22 is also conjugated to a DIG linker moiety and its binding affinity is evaluated. Data for these assays are plotted in FIG. 6. The peptide homodimers in these binding assays exhibit TRAIL R2 binding affinities between 1,000 and 20,000 times greater than the binding affinities of the corresponding peptide monomers. Specifically, the peptide homodimer based on the monomer amino acid sequence of SEQ ID NO: 33 has in these binding assays a binding activity (IC₅₀) having a range of about 25 pM to about 92 pM, while the peptide monomer based on the amino acid sequence of SEQ ID NO: 33 has in these binding assays an activity (IC₅₀) having a range of about 0.2 μM to about 5.7 μM. Furthermore, in these assays, the peptide homodimer based on the monomer amino acid sequence of SEQ ID NO: 22 has in these assays a binding activity (IC₅₀) of 6.2 nM while the peptide monomer based on the amino acid sequence of SEQ ID NO: 22 has in these assays a binding activity (IC₅₀) having a range of about 3.55 μM and about 10.7 μM. The presence of a DIG linker moiety above does not produce any significant change in binding activity for the peptide monomer of SEQ ID NO: 22, which has in these assays a binding activity (IC₅₀) having a range of about 1.2 μM to about 2.3 μM. Hence, the multimerization, including dimerization, of TRAIL agonist peptides has the ability to greatly enhance binding affinity.

To investigate what effect positional placement of the linker moiety may have on binding affinity, dimer constructs are generated based on the agonist peptide sequence of SEQ ID NO: 21 (AcWDCLDN(X3)IGRRQCVKL-NH2) in which a lysine residue conjugated to a linker moiety is positioned at different locations along the amino acid sequence. A first peptide dimer construct is thus generated based on the monomer amino acid sequence of SEQ ID NO: 19, in which the lysine residue and linker moiety are near the C-terminus of the amino acid sequence. A second peptide dimer construct is also generated, based on the monomer amino acid sequence of SEQ ID NO: 33, in which the lysine residue is near the center of the amino acid sequence. These constructs are illustrated schematically in FIG. 7.

Investigation of the constructs' binding affinities in several of the AlphaQuest® binding competition assays indicates that the peptide homodimer based on the monomer amino acid sequence of SEQ ID NO: 19 (See, FIG. 7B) binds TRAIL R2 with a binding activity (IC₅₀) of about 7 μM and has a range of about 1 μM to about 56 μM, and the peptide homodimer based on the monomer amino acid sequence of SEQ ID NO: 33 (See, FIG. 7A) binds TRAIL R2 with a binding activity (IC₅₀) of about 61 pM and has a range of about 25 pM to about 92 pM.

The apoptotic activity of the different constructs is also evaluated, using an HCT-116 proliferation assay and a Jurkat proliferation assay, described above. The peptide dimer construct based on the monomer amino acid sequence of SEQ ID NO: 19 (See, FIG. 7B) exhibits apoptotic activity having a range of about 0.74 μM to about 2.2 μM in the HCT-116 proliferation assay and an apoptotic activity having a range of about 0.5 μM to about 7.0 μM in the Jurkat proliferation assay. No apoptotic activity is detected for the peptide dimer construct based on the monomer amino acid sequence of SEQ ID NO: 33 (See, FIG. 7A). Hence, positioning of a linker moiety joining peptide monomers of the invention (e.g., in a peptide dimer construct) can independently affect both binding affinity and apoptotic activity.

1.8 Synthesis of TRAIL R2 Agonist Trimers

Trimer constructs of TRAIL R2 agonist peptides are also prepared to evaluate the effect of trimeric multimerization on binding activity and apoptotic activity. More specifically, peptide monomers of the invention are joined into trimeric constructs using a Tris-succinimidyl aminotriacetate (TSAT) linker moiety as illustrated in FIG. 8A.

Synthetic peptides are prepared using Fmoc chemistry on TentaGel R RAM (0.18 mmol/g, 0.4 g; particle size 90 um) from Rapp Polymere GmbH (Tubingen, Germany) resins using standard DIC/HOBt batchwise solid-phase synthesis protocols on a PTI Symphony peptide synthesizer. The N-terminal Fmoc-group is removed with 20% piperidine in DMF, and the N-terminal amine is capped with a mixture of acetic anhydride/pyridine/THF. Following resin and side-chain cleavage with 85% TFA/10% triisopropylsilane/2.5% H20/2.5% thioanisole, the crude peptides are precipitated with cold diethyl ether and washed twice with ether; material is solubilized in a mixture of 10% DMSO/40% acetonitrile/50% NH₄OAc buffer (10 mM) at a peptide concentration of 1 mg/mL for oxidation of the cysteines. The oxidation is monitored by RP-HPLC and LCMS. Once the oxidation is complete (2-12 h depending on the sequence), the peptides are concentrated, diluted with 10% acetonitrile in water, and purified by preparative C₁₈ RP-HPLC using linear gradients of acetonitrile (containing 0.1% TFA) in H20 (containing 0.1% TFA) on either a Waters RCM Delta-Pak, 300 Å, 15 μm, 25×200 mm column or XTerra Prep MS, 125 Å, 5 μm, 19×50 mm column. Trimerization is accomplished by dissolution of peptide monomer in DMF, followed by addition of 10 eq. DIEA and portion-wise addition of 0.33 eq. Tris-succinimidyl aminotriacetate (TSAT). The reaction is monitored by HPLC and LCMS. Upon completion, the reaction mixture is diluted with water and purified by preparative C₁₋₈ RP-HPLC using the same buffer conditions and columns as for the peptide monomers. Final products are analyzed by analytical C₁₈ RP-HPLC (Zorbax SB, 3.5 μm, 2.1×75 mm) with a gradient of 20-50% CH₃CN in aqueous 0.1% TFA. An exemplary peptide trimer based on the monomer amino acid sequence of SEQ ID NO: 19 is illustrated in FIG. 8B.

### Example 2

### TRAIL R2 Antagonist Peptides

2.1 Competition Binding Assay

The binding affinity of the antagonist peptides and peptide-based compounds of the invention are measured according to the AlphaQuest® Competitive Binding Assay, described above, in Example 1.1. The IC₅₀ values are determined for each test peptide or test compound. The compounds tested are homodimers and homotrimers of peptides comprising the amino sequence set forth in SEQ ID NOs: 33 and 34, linked by DIG or TSAT, respectively. The type of multimer (e.g., dimer or trimer, etc.), sequence, type of linker, and binding affinity for each compound tested is summarized in Table 6, below.

TABLE 6

| SEQ ID | | | | | | | | | | | | | | | | | | | | | Binding |
|---|---|---|---|---|---|---|---|---|---|---|---|---|---|---|---|---|---|---|---|---|---|
| NO. | Construct | Antagonist Peptide Sequences | | | | | | | | | | | | | | | | | | Linker | (IC50, pM) |
| 33 | Dimer | Ac | W | D | C* | L | D | N | R | I | G | K^{†} | R | Q | C* | V | R | | NH2 | DIG | 111 |
| 34 | Dimer | Ac | W | D | C* | L | D | N | R | I | G | K^{†} | R | Q | C* | V | R | A | NH2 | DIG | 60 |
| 33 | Trimer | Ac | W | D | C* | L | D | N | R | I | G | K^{†} | R | Q | C* | V | R | A | NH2 | TSAT | 38 |

^{†}= Site of linker attachment
*= Cysteine residue of disulfide bond

2.2 Jurkat Antagonist Assay

In order to test the activity of the antagonist peptides and peptide-based compounds of the invention, the ability of the test peptides to inhibit TRAIL ligand-induced apoptosis of Jurkat cells is measured. Jurkat cells are resuspended at a density of 1×10⁶ cells/mL in Growth Medium, and 100 μl cells is added to each well of the assay plates. Serial dilutions of synthetic peptides are prepared using Growth Medium in a 96-well Deep-Well round bottom tissue culture plate, and 11 μL of the serially diluted peptides is added to each well of the assay plates. The plate is incubated for 45 minutes in a 37° C., 5% CO₂ incubator. 50 ng/mL TRAIL ligand (Axxora, San Diego, Calif.), diluted in Growth Medium, is added to each well of the assay plate. The plates are incubated in a 37° C., 5% CO₂ incubator for 16-20 hours. Proliferation is assessed by adding 10 μL of MTT stock solution (ATCC, Manassas, Va.) to the wells. The plates are incubated in a 37° C., 5% CO₂ incubator for another 2-4 hours, followed by the addition of 100 μL of 20% SDS with 0.01 N HCl. After incubation overnight in a 37° C., 5% CO₂ incubator, the plates are quantified by reading at OD₅₉₅ in a SpectraMax M5 microplate reader (Molecular Devices, Sunnyvale, Calif.). The data are analyzed using GraphPad Prism software (La Jolla, Calif.) to calculate EC₅₀ values from a 4-parameter logistic equation.

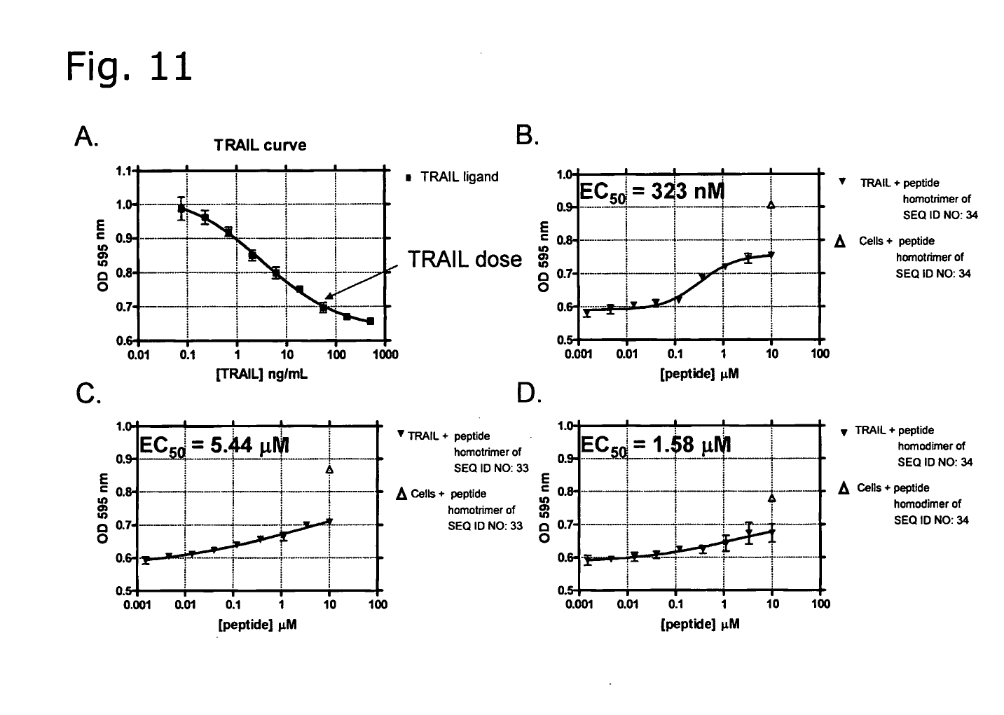

First, using this assay, a “TRAIL curve” is generated showing the increasing induction of Jurkat cell apoptosis by addition of TRAIL R2 ligand (TRAIL ligand) as the concentration of TRAIL ligand is increased. An exemplary TRAIL curve is illustrated in FIG. 11A. Next, using the TRAIL curve, an optimal concentration of TRAIL ligand control is selected (e.g., 100 ng/ml) for use in the Jurkat antagonist assay (i.e., the concentration at which Jurkat cell apoptosis is near maximal) to test the antagonist peptide compounds of the invention. In FIGS. 11B-D, exemplary half-maximal effective concentrations (EC₅₀) of antagonist peptides needed to restore Jurkat cell proliferation (i.e., inhibit TRAIL ligand induction of apoptosis) are determined from Jurkat antagonist assays for a peptide trimer comprising the monomer amino acid sequence set forth in SEQ ID NO: 34 (EC₅₀=323 nM), a peptide dimer comprising the monomer amino acid sequence set forth in SEQ ID NO: 33 (EC₅₀=5.44 μM), and a peptide dimer comprising the monomer amino acid sequence set forth in SEQ ID NO: 34 (EC₅₀=1.58 μM). Hence, antagonist peptide trimers and dimers of the invention can inhibit TRAIL ligand-induced apoptosis activity.

### CONCLUSION

These examples demonstrate that novel peptides specific for the TRAIL R2 receptor can be identified through synthetic peptide screening. Dimerization led to apoptotic activity in whole cell assays of agonist peptides (See, FIG. 6), and dimers of antagonist peptides inhibited the ability of TRAIL ligand to induce apoptosis in Jurkat cells (See, FIGS. 11B-D). Optimization of the original hit peptide via truncations and architectural modifications of SEQ ID NO: 4 can enhance the binding affinity up to 10,000-fold (See, FIGS. 2 and 6), and trimerization increases apoptotic activity of peptide agonist trimers by as much as 5-fold over the corresponding peptide agonist dimer (See, FIG. 9). The foregoing examples also demonstrate usefulness of the peptides of the present invention as mimetics of protein targets. Furthermore, peptide-based drugs may provide superior product profiles over therapeutic proteins. Advantages of peptide-based drugs can include reduced immunogenicity, reduced dosing frequency, flexible storage and uncomplicated chemical synthesis.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Numerous references, including various patents, patent publications and non-patent documents, are cited and discussed in the description of the invention. The citation or discussion of such references is provided merely to clarify the description of the present invention and is not an admission that any such reference is “prior art” to any invention described herein. All references cited and discussed in this specification are hereby incorporated by reference in their entirety and to the same extent as if each reference was individually incorporated by reference.

Throughout this specification and claims, the word “comprise,” or variations such as “comprises” or “comprising,” will be understood to imply the inclusion of a stated integer or group of integers but not the exclusion of any other integer or group of integers.

We claim:

1. A compound comprising a peptide that binds to a TRAIL R2 receptor and comprises a sequence of amino acids Ac-W-D-C-L-D-N-X1-I-G-R-R-Q-C-V-X2-L-NH₂ (SEQ ID NO: 18), wherein each amino acid is indicated by standard one letter abbreviation, and wherein X1 and X2 are each independently selected from the amino acid residues arginine (R) and lysine (K).

2. A compound comprising a peptide that binds to a TRAIL R2 receptor and comprises a sequence of amino acids selected from the group consisting of

| | |
|---|---|
| AcWDCLDNRIGRRQCVKL-NH2; | (SEQ ID NO: 19) |
| AcGGSWDCLDNRIGRRQCVKL-NH2; | (SEQ ID NO: 20) |
| AcWDCLDN(X3)IGRRQCVKL-NH2; | (SEQ ID NO: 21) |
| AcWDCLDRPGRRQCVK-NH2; | (SEQ ID NO: 22) |
| AcWDCLDNKIGRRQCVRL-NH2; | (SEQ ID NO: 23) |
| AcCLDNRIGRRQCV; | (SEQ ID NO: 24) |
| AcDCLDNRIGRRQCVKL-NH2; | (SEQ ID NO: 25) |
| AcWDCLDNRIGKRQCVRL-NH2; | (SEQ ID NO: 26) |
| AcWDCLDNRIG(X4)RQCV(X5)L-NH2; | (SEQ ID NO: 27) |
| AcWDCLDNRIGRRQCVK-NH2; | (SEQ ID NO: 28) |
| AcWDCLVDRPGRRQCVRLEK-NH2; | (SEQ ID NO: 29) |
| AcWDCLVDRPGRRQCVRLERK-NH2; | (SEQ ID NO: 30) |
| AcWDCLVDRPGRRQCVKLER-NH2; | (SEQ ID NO: 31) |
| GGGSWDCLDNRIGRRQCVKL; | (SEQ ID NO: 4) |
| AcCWDLDNRIGRRQVCKL-NH2; | (SEQ ID NO: 36) |
| and | |
| GGGSWDCLDNRIGRRQCVKL-NH2 | (SEQ ID NO: 32) |

wherein each amino acid is indicated by standard one letter abbreviation, and wherein X3, X4, and X5 are independently selected from the amino acid residues arginine (R) and lysine (K).

3. A compound comprising a peptide that binds to a TRAIL R2 receptor and comprises a sequence of amino acids:

| | |
|---|---|
| AcWDCLDNRIGKRQCVR-NH2; | (SEQ ID NO: 33) |
| or | |
| AcWDCLDNRIGKRQCVRA-NH2. | (SEQ ID NO: 34) |

4. The compound of claim 1, 2, or 3, wherein said peptide is a monomer.

5. The compound of claim 1, 2, or 3, wherein said peptide is a dimer.

6. The compound of claim 1, 2, or 3, wherein said peptide is a homodimer.

7. The compound of claim 1, 2, or 3, wherein said peptide is a trimer.

8. The compound of claim 1, 2, or 3, wherein said peptide is a homotrimer.

9. The compound of claim 1, 2, or 3, wherein said peptide is a dimer further comprising a linker.

10. The compound of claim 1, 2, or 3, wherein the first amino acid residue of said peptide is acetylated.

11. The compound of claim 9, wherein the linker is diglycolic acid (DIG) or Tris-succinimidyl aminotriacetate (TSAT).

12. A compound comprising a peptide trimer that binds to a TRAIL R2 receptor and where each peptide comprises a sequence of amino acids Ac-W-D-C-L-D-N-R-I-G-R-R-Q-C-V-K-L-NH₂ (SEQ ID NO: 19), wherein each amino acid is indicated by standard one letter abbreviation and AcW is N-acetyl-tryptophan.

13. A method for treating cancer in a patient, which method comprises administering to the patient a therapeutically effective amount of the compound of claim 1, 2, or 3.

14. A method for treating asthma in a patient, which method comprises administering to the patient a therapeutically effective amount of the compound of claim 1, 2, or 3.

15. A pharmaceutical composition comprising the compound of claim 1, 2, or 3 and a pharmaceutically acceptable carrier.

16. A compound that binds to and activates a TRAIL R2 receptor, which compound comprises a peptide dimer of SEQ ID NO: 19 having the formula: 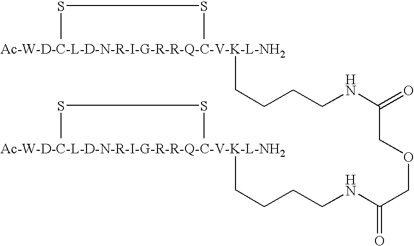
wherein
(i) in each peptide monomer of the peptide dimer, each amino acid is indicated by standard one letter abbreviation and AcW is N-acetyl-tryptophan; and
(ii) each peptide monomer of the peptide dimer contains an intramolecular disulfide bond between the two cysteine (C) residues of each peptide monomer.

17. A compound that binds to and activates a TRAIL R2 receptor, which compound comprises a peptide trimer of SEQ ID NO: 19 having the formula: 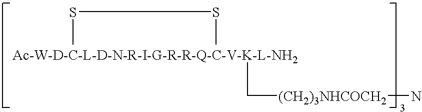
wherein
(i) in each peptide monomer of the peptide trimer, each amino acid is indicated by standard one letter abbreviation and AcW is N-acetyl-tryptophan; and
(ii) each peptide monomer of the peptide trimer contains an intramolecular disulfide bond between the two cysteine (C) residues of each peptide monomer.

18. A method for treating cancer in a patient, which method comprises administering to the patient a therapeutically effective amount of the compound of claim 16 or 17.

19. A pharmaceutical composition comprising the compound of claim 16 or 17 and a pharmaceutically acceptable carrier.

20. A compound that binds to and antagonizes a TRAIL R2 receptor, which compound comprises a peptide trimer of SEQ ID NO: 34 having the formula:

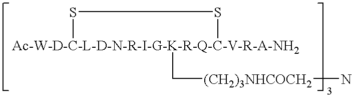

21. A method for treating an asthma related disorder in a patient, which method comprises administering to the patient a therapeutically effective amount of the compound of claim 20.

22. A pharmaceutical composition comprising the compound of claim 20 and a pharmaceutically acceptable carrier
